(19) United States
(12) Patent Application Publication
(10) Pub. No.: US 2013/0317056 A1
(43) Pub. Date: Nov. 28, 2013

(54) NEURONAL NICOTINIC AGONISTS AND METHODS OF CORRELATING COMT SNPS

(71) Applicant: ABBVIE INC., North Chicago, IL (US)

(72) Inventors: George M. Haig, Gurnee, IL (US); Jeffrey F. Waring, Franklin, WI (US); Chih-Hung Lee, Vernon Hills, IL (US); Deli Wang, Wilmette, IL (US)

(73) Assignee: AbbVie Inc., North Chicago, IL (US)

(21) Appl. No.: 13/826,532

(22) Filed: Mar. 14, 2013

Related U.S. Application Data
(60) Provisional application No. 61/651,431, filed on May 24, 2012.

(51) Int. Cl. C12Q 1/68

(52) U.S. Cl. CPC C12Q 1/6883
USPC 514/294; 435/6.11; 514/305

(57) ABSTRACT

This application is directed to a-neuronal nicotinic receptor agonists selective for α7-subtype that are useful for improving cognition impairment in patients having schizophrenia, a schizophreniform disorder or a related schizophrenia spectrum psychotic disorder. Compounds and compositions containing such compounds, and methods of using such compound and compositions are described herein.

### CROSS-REFERENCE TO RELATED APPLICATIONS

This application claims the benefit of U.S. Application No. 61/651,431, filed on May 24, 2012, the contents of which are herein incorporated by reference.

### FIELD OF THE INVENTION

The present disclosure is directed to a-neuronal nicotinic receptor agonists selective for α7-subtype that are useful for improving cognition impairment in patients having schizophrenia, a schizophreniform disorder or a related schizophrenia spectrum psychotic disorder. Compounds and compositions containing such compounds, and methods of using such compound and compositions are described herein.

### BACKGROUND OF THE INVENTION

Nicotinic acetylcholine receptors (nAChRs) are widely distributed throughout the central (CNS) and peripheral (PNS) nervous systems. Such receptors play an important role in regulating CNS function, particularly by modulating release of a wide range of neurotransmitters, including, but not necessarily limited to, acetylcholine, norepinephrine, dopamine, serotonin, and GABA. The α7 and α4β2 nAChRs have been shown to play a significant role in enhancing cognitive function, including aspects of learning, memory and attention. For example, α7 nAChRs have been linked to conditions and disorders related to attention deficit disorder, attention deficit hyperactivity disorder (ADHD), schizophrenia, Alzheimer's disease (AD), mild cognitive impairment, senile dementia, dementia associated with Lewy bodies, dementia associated with Down's syndrome, AIDS dementia, and Pick's disease, as well as inflammation. The α4β2 receptor subtype is implicated in attention, cognition, epilepsy, and pain control as well as smoking cessation or nicotine withdrawal syndrome.

The activity at both α7 and α4β2 nAChRs can be modified or regulated by the administration of subtype selective nAChR ligands. The ligands can exhibit antagonist, agonist, or partial agonist properties. Compounds that function as allosteric modulators are also known. Although compounds that nonselectively demonstrate activity at a range of nicotinic receptor subtypes including α 7 nAChRs are known, it would be beneficial to provide compounds that interact selectively with α 7-containing neuronal nAChRs compared to other subtypes.

Since the α7 and α4β2 nAChRs are involved in so many biological pathways and responses, it would be beneficial to identify other genes that are affected by α7 and α4β2 nAChRs modulation. Therefore, there is a need in the art to identify genes and genetic variations related to these genes that will assist clinicians in better identifying and using particular α7 and α4β2 nAChRs modulators for treatment and assessment of treatment regimens for improving symptoms associated with nAChR-mediated conditions such as schizophrenia and other related disorders. These methods will provide clinicians with another tool for identifying neuronal nicotinic acetylcholine receptor modulators (agonist, antagonists) that treats such conditions in a safe and efficacious manner.

### SUMMARY OF INVENTION

In one aspect, the present invention relates to a method of identifying a patient with schizophrenia as a candidate for effective treatment with a nicotinic acetylcholine receptor ligand modulator, the method. This method comprises the steps of:
(a) obtaining a sample from the patient with schizophrenia;
(b) determining the identity of an allele of at least one single nucleotide polymorphism (SNP) locus associated with the catechol-O-methyltransferase (COMT) gene in the sample;
(c) determining the smoking status of the patient with schizophrenia;
(d) identifying the patient with schizophrenia as a candidate for effective treatment with the nicotinic alpha 7 receptor agonist based on the presence or absence of a particular SNP allele associated with the COMT gene in the sample and the smoking status of the patient with schizophrenia; and
(e) administering to the patient in need thereof an effective amount of a particular nicotinic acetylcholine receptor ligand modulator based upon result of step (d).

In the above method, the nicotinic acetylcholine receptor is α-7 nicotinic receptor. Additionally, in the above method, the presence of at least one SNP allele associated with the COMT gene in the patient with schizophrenia identifies the patient with schizophrenia as a candidate for effective treatment with the nicotinic alpha 7 receptor agonist.

In another aspect, the present invention relates to a method of identifying a patient with schizophrenia with an increased likelihood of response to treatment with a nicotinic alpha 7 receptor agonist. The method comprises the steps of:
(a) obtaining a sample from the patient with schizophrenia;
(b) determining the identity of an allele of at least one single nucleotide polymorphism (SNP) locus associated with the catechol-O-methyltransferase (COMT) gene in the sample;
(c) determining the smoking status of the patient with schizophrenia; and
(d) identifying the patient with schizophrenia as having an increased likelihood of response to treatment with the nicotinic alpha 7 receptor agonist based on the presence or absence of a particular SNP allele associated with the COMT gene in the sample and the smoking status of the patient with schizophrenia.

In the above method, the presence of at least one SNP allele associated with the COMT gene in the patient with schizophrenia identifies the patient with schizophrenia as a candidate for effective treatment with the nicotinic alpha 7 receptor agonist.

In another aspect, the present invention relates to a method of identifying and treating a patient with schizophrenia with an effective dosage of nicotinic alpha 7 receptor agonist. The method comprises the steps of:
(a) obtaining a sample from the patient with schizophrenia;
(b) determining the identity of an allele of at least one single nucleotide polymorphism (SNP) locus associated with the catechol-O-methyltransferase (COMT) gene in the sample;
(c) determining the smoking status of the patient with schizophrenia;
(d) identifying the patient with schizophrenia as a candidate for effective treatment with the nicotinic alpha 7 receptor agonist based on the presence or absence of a particular SNP allele associated with the COMT gene in the sample and the smoking status of the patient with schizophrenia; and
(e) administering an effective dosage of nicotinic alpha 7 receptor agonist to the patient with schizophrenia identified as being a candidate for effective treatment with the nicotinic alpha 7 receptor agonist.

In any of the above-described methods, the SNP associated with the COMT gene is located in the COMT gene or in a region surrounding the COMT gene. Alternatively, in any of the above-described methods, the SNP associated with the COMT gene is located in the COMT gene.

Moreover, in any of the above-described methods, the SNP is at least one of rs6269, rs4633, rs4680, and rs4818, or a SNP in linkage disequilibrium with at least one of the foregoing SNPs, or combinations thereof.

Still further, in any of the above-described methods, the SNP is at least one of rs6269, rs4633, rs4680, and rs4818, or combinations thereof.

Yet still further in any of the above-described methods, the SNP is at least one of a SNP in linkage disequilibrium with at least one of rs6269, rs4633, rs4680, and rs4818, or combinations thereof.

Still even further in any of the above-described methods, the presence of at least one of G/C or G/G for rs4818, A/A or G/A or rs4680, T/T or C/T for rs4633, G/G or A/G for rs6269 identifies the patient with schizophrenia as a candidate for effective treatment with nicotinic alpha 7 receptor agonist.

Still even further in any of the above-described methods, the patient with schizophrenia is a smoker.

Still even further in any of the above-described methods, the patient with schizophrenia is a non-smoker.

Yet still even further in any of the above described methods, the nicotinic alpha 7 receptor agonist comprises a compound selected from the group consisting of (4s)-4-(5-phenyl-1,3,4-thiadiazol-2-yloxy)-1-azatricyclo[3.3.1.1]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide and salts thereof.

Still even further in any of the above-described methods, the nicotinic alpha 7 receptor agonist comprises (4s)-4-(5-phenyl-1,3,4-thiadiazol-2-yloxy)-1-azatricyclo[3.3.1.1^{3,7}]decane.

Still even further in any of the above-described methods, the effective dosage range of the nicotinic alpha 7 receptor agonist is about 10-25 mg/kg of body weight daily.

In still yet another aspect, the present invention relates to a method for monitoring the treatment of a patient suffering from schizophrenia, schizophreniform disorder or a related schizophrenia spectrum psychotic disorder. The method comprises the steps of:
(a) obtaining a sample from the patient wherein the patient is already under a treatment regimen of a particular nAChr ligand modulator;
(b) determining the identity of an allele of at least one single nucleotide polymorphism (SNP) locus associated with the COMT gene in the sample;
(c) determining the smoking status of the patient; and if necessary,
(d) modifying the course of treatment including administering a different nAChr ligand modulator based upon the presence or absence of particular SNPs associated with the COMT gene in the patient.

In the above-described method, the SNP associated with the COMT gene is located in the COMT gene or in a region surrounding the COMT gene. Alternatively, in the above-described method, the SNP associated with the COMT gene is located in the COMT gene.

Moreover, in the above-described method, the SNP is at least one of rs6269, rs4633, rs4680, and rs4818, or a SNP in linkage disequilibrium with at least one of the foregoing SNPs, or combinations thereof.

Still further, in the above-described method, the SNP is at least one of rs6269, rs4633, rs4680, and rs4818, or combinations thereof.

Yet still further in the above-described method, the SNP is at least one of a SNP in linkage disequilibrium with at least one of rs6269, rs4633, rs4680, and rs4818, or combinations thereof.

Still even further in the above-described method, the presence of at least one of G/C or G/G for rs4818, A/A or G/A or rs4680, T/T or C/T for rs4633, G/G or A/G for rs6269 identifies the patient with schizophrenia as a candidate for effective treatment with nicotinic alpha 7 receptor agonist.

Still even further in the above-described method, the patient with schizophrenia is a smoker.

Still even further in the above-described method, the patient with schizophrenia is a non-smoker.

Yet still even further in any of the above described methods, the nicotinic alpha 7 receptor agonist comprises a compound selected from the group consisting of (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide and salts thereof.

Still even further in the above-described method, the nicotinic alpha 7 receptor agonist comprises (4s)-4-(5-phenyl-1,3,4-thiadiazol-2-yloxy)-1-azatricyclo[3.3.1.1^{3,7}]decane.

Still even further in the above-described method, the effective dosage range of the nicotinic alpha 7 receptor agonist is about 10-25 mg/kg of body weight daily.

Additional aspects of the invention and further details are provided herein.

### BRIEF DESCRIPTION OF THE DRAWINGS

FIG. 1 is a table showing the MCCB composite scores for placebo, Compound A 10 mg and Compound A 25 mg dose groups, with data shown according to cigarette smoking status (current smoker or current nonsmoker).

FIG. 2 graphically depicts the mean change from baseline as measured by the MATRICS Consensus Cognitive Battery (MCCB) in patients administered (4s)-4-(5-phenyl-1,3,4-thiadiazol-2-yloxy)-1-azatricyclo[3.3.1.1^{3,7}]decane (Compound A; “Comp A”) in stable subjects with schizophrenia receiving their antipsychotic treatment regimen in a double-blind, parallel-group Phase 2a clinical study when compared with placebo. In addition to the baseline, data are presented at both week 6 and week 12.

FIG. 3 graphically depicts COMT activity over a wide range of protein concentrations (top) and incubation times (bottom). COMT activity is represented by femtomoles of [³H]-methylcatechol produced in the assay per 20 minutes (fmol [³H]-methylcatechol/20 min), plotted vs. μg protein. The data illustrates that the assay has a very wide dynamic range for determination of COMT activity for a range of protein values and for incubation times.

FIG. 4 graphically depicts COMT activity in mouse brain frontal cortex in the C57BL/6J mouse strain and the C57L/J strain. COMT activity is represented as fmol methylcatechol/20 min, with data points shown for each mouse strain. The data demonstrates that there is a 1.5-fold greater specific activity for COMT in the C57BL/6J mouse strain vs. the C57L/J strain.

FIG. 5 graphically depicts COMT activity in both mouse frontal cortex (FIG. 5A) and washed erythrocytes (FIG. 5B), in the C57BL/6J mouse strain and the C57L/J strain. COMT activity is represented as fmol [³H]-methylcatechol/20 min, with data points shown for each mouse strain. The data demonstrates that strain differences between C57BL/6J and C57L/J can be detected in multiple tissues.

### DETAILED DESCRIPTION

The present invention relates to the use of a nicotinic acetylcholine receptor (nAChR) ligand modulator for the preparation of a medicament for improving symptoms of cognitive deficit associated schizophrenia in a patient. The method may also be applicable to monitoring the effectiveness of a nAChR ligand. Specifically, the present method has identified a relationship between a therapeutic response to nicotinic acetylcholine receptor (nAChR) ligand modulator and genetic variants (i.e. polymorphism) of the catechol-O-methyltransferase (COMT) gene in schizophrenic individuals. The method may be further applied by associating particular nAChR ligands with particular genetic variations of the COMT gene for both effective implementation and monitoring of therapies for schizophrenic individuals. The therapeutic effectiveness of the nAChR ligand may further be affected by whether the individual is a smoker or a non-smoker.

### 1. Definitions

As used throughout this specification and the claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene” means a divalent group derived from a straight or branched chain hydrocarbon of from 2 to 10 carbon atoms containing at least one double bond. Representative examples of alkenylene include, but are not limited to, —CH═CH—, —CH═CH₂CH₂—, and —CH═C(CH₃)CH₂—.

The term “alkenyloxy” as used herein, means an alkenyl group, as defined herein, appended to the parent molecular moiety through an oxygen atom. Representative examples of alkenyloxy include, but are not limited to, allyloxy, 2-butenyloxy and 3-butenyloxy.

The term “alkoxy” as used herein, means an alkyl group, as defined herein, appended to the parent molecular moiety through an oxygen atom. Representative examples of alkoxy include, but are not limited to, methoxy, ethoxy, prop oxy, 2-prop oxy, butoxy, tert-butoxy, pentyloxy, and hexyloxy.

The term “alkoxyalkoxy” as used herein, means an alkoxy group, as defined herein, appended to the parent molecular moiety through another alkoxy group, as defined herein. Representative examples of alkoxyalkoxy include, but are not limited to, tert-butoxymethoxy, 2-ethoxyethoxy, 2-methoxyethoxy, and methoxymethoxy.

The term “alkoxyalkoxyalkyl” as used herein, means an alkoxyalkoxy group, as defined herein, appended to the parent molecular moiety through an alkyl group, as defined herein.

Representative examples of alkoxyalkoxyalkyl include, but are not limited to, tert-butoxymethoxymethyl, ethoxymethoxymethyl, (2-methoxyethoxy)methyl, and 2-(2-methoxyethoxy)ethyl.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxycarbonylalkyl” as used herein, means an alkoxycarbonyl group, as defined herein, appended to the parent molecular moiety through an alkyl group, as defined herein. Representative examples of alkoxycarbonylalkyl include, but are not limited to, 3-methoxycarbonylpropyl, 4-ethoxycarbonylbutyl, and 2-tert-butoxycarbonylethyl.

The term “alkoxysulfonyl” as used herein, means an alkoxy group, as defined herein, appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonylalkyl” as used herein, means an alkylcarbonyl group, as defined herein, appended to the parent molecular moiety through an alkyl group, as defined herein. Representative examples of alkylcarbonylalkyl include, but are not limited to, 2-oxopropyl, 3,3-dimethyl-2-oxopropyl, 3-oxobutyl, and 3-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylene” means a divalent group derived from a straight or branched chain hydrocarbon of from 1 to 10 carbon atoms. Representative examples of alkylene include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfinylalkyl” as used herein, means an alkylsulfinyl group, as defined herein, appended to the parent molecular moiety through an alkyl group, as defined herein. Representative examples of alkylsulfinylalkyl include, but are not limited to, methylsulfinylmethyl and ethylsulfinylmethyl.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylsulfonylalkyl” as used herein, means an alkylsulfonyl group, as defined herein, appended to the parent molecular moiety through an alkyl group, as defined herein. Representative examples of alkylsulfonylalkyl include, but are not limited to, methylsulfonylmethyl and ethylsulfonylmethyl.

The term “alkylthio” as used herein, means an alkyl group, as defined herein, appended to the parent molecular moiety through a sulfur atom. Representative examples of alkylthio include, but are not limited to, methylthio, ethylthio, tert-butylthio, and hexylthio.

The term “alkylthioalkyl” as used herein, means an alkylthio group, as defined herein, appended to the parent molecular moiety through an alkyl group, as defined herein. Representative examples of alkylthioalkyl include, but are not limited to, methylthiomethyl and 2-(ethylthio)ethyl.

The term “alkynyl” as used herein, means a straight or branched chain hydrocarbon group containing from 2 to 10 carbon atoms and containing at least one carbon-carbon triple bond. Representative examples of alkynyl include, but are not limited to, acetylenyl, 1-propynyl, 2-propynyl, 3-butynyl, 2-pentynyl, and 1-butynyl.

The term “alkynylene” means a divalent group derived from a straight or branched chain hydrocarbon of from 2 to 10 carbon atoms containing at least one triple bond. Representative examples of alkynylene include, but are not limited to, —C≡C—, —CH₂C≡C—, —CH(CH₃)CH₂C≡C—, —C≡CCH₂—, and —C≡CCH(CH₃)CH₂—.

The term “alkynyloxy” as used herein, means an alkynyl group, as defined herein, appended to the parent molecular moiety through an oxygen atom. Representative examples of alkynyloxy include, but are not limited to, 2-propynyloxy and 2-butynyloxy.

The term “aryl,” as used herein, means phenyl, a bicyclic aryl or a tricyclic aryl. The bicyclic aryl is naphthyl, a phenyl fused to a cycloalkyl, or a phenyl fused to a cycloalkenyl. Representative examples of the bicyclic aryl include, but are not limited to, dihydroindenyl, indenyl, naphthyl, dihydronaphthalenyl, and tetrahydronaphthalenyl. The tricyclic aryl is anthracene or phenanthrene, or a bicyclic aryl fused to a cycloalkyl, or a bicyclic aryl fused to a cycloalkenyl, or a bicyclic aryl fused to a phenyl. Representative examples of tricyclic aryl ring include, but are not limited to, azulenyl, dihydroanthracenyl, fluorenyl, and tetrahydrophenanthrenyl.

The aryl groups of this invention can be substituted with 1, 2, 3, 4 or 5 substituents independently selected from alkenyl, alkoxy, alkoxyalkoxy, alkoxyalkoxyalkyl, alkoxyalkyl, alkoxycarbonyl, alkoxycarbonylalkyl, alkyl, alkylcarbonyl, alkylcarbonylalkyl, alkylcarbonyloxy, alkylsulfinyl, alkylsulfinylalkyl, alkylsulfonyl, alkylsulfonylalkyl, alkylthio, alkylthioalkyl, alkynyl, carboxy, carboxyalkyl, cyano, cyanoalkyl, formyl, formylalkyl, halogen, haloalkyl, hydroxy, hydroxyalkyl, mercapto, nitro, —NZ₁Z₂, and (NZ₃Z₄)carbonyl.

The term “arylalkoxy” as used herein, means an aryl group, as defined herein, appended to the parent molecular moiety through an alkoxy group, as defined herein. Representative examples of arylalkoxy include, but are not limited to, 2-phenylethoxy, 3-naphth-2-ylpropoxy, and 5-phenylpentyloxy.

The term “arylalkoxycarbonyl” as used herein, means an arylalkoxy group, as defined herein, appended to the parent molecular moiety through a carbonyl group, as defined herein. Representative examples of arylalkoxycarbonyl include, but are not limited to, benzyloxycarbonyl and naphth-2-ylmethoxycarbonyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylalkylthio” as used herein, means an arylalkyl group, as defined herein, appended to the parent molecular moiety through a sulfur atom. Representative examples of arylalkylthio include, but are not limited to, 2-phenylethylthio, 3-naphth-2-ylpropylthio, and 5-phenylpentylthio.

The term “arylcarbonyl” as used herein, means an aryl group, as defined herein, appended to the parent molecular moiety through a carbonyl group, as defined herein. Representative examples of arylcarbonyl include, but are not limited to, benzoyl and naphthoyl.

The term “aryloxy” as used herein, means an aryl group, as defined herein, appended to the parent molecular moiety through an oxygen atom. Representative examples of aryloxy include, but are not limited to, phenoxy, naphthyloxy, 3-bromophenoxy, 4-chlorophenoxy, 4-methylphenoxy, and 3,5-dimethoxyphenoxy.

The term “aryloxyalkyl” as used herein, means an aryloxy group, as defined herein, appended to the parent molecular moiety through an alkyl group, as defined herein. Representative examples of aryloxyalkyl include, but are not limited to, 2-phenoxyethyl, 3-naphth-2-yloxypropyl and 3-bromophenoxymethyl.

The term “arylthio” as used herein, means an aryl group, as defined herein, appended to the parent molecular moiety through a sulfur atom. Representative examples of arylthio include, but are not limited to, phenylthio and 2-naphthylthio.

The term “arylthioalkyl” as used herein, means an arylthio group, as defined herein, appended to the parent molecular moiety through an alkyl group, as defined herein. Representative examples of arylthioalkyl include, but are not limited to, phenylthiomethyl, 2-naphth-2-ylthioethyl, and 5-phenylthiomethyl.

The term “AUC_{∞}” refers to the area under the plasma concentration time curve (AUC) extrapolated to infinity.

The term “azido” as used herein, means a —N₃ group.

The term “carbonyl” as used herein, means a —C(O)— group.

The term “carboxy” as used herein, means a —CO₂H group.

The term “carboxyalkyl” as used herein, means a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkenyl” as used herein, means a cyclic hydrocarbon containing from 3 to 8 carbons and containing at least one carbon-carbon double bond formed by the removal of two hydrogens. Representative examples of cycloalkenyl include, but are not limited to, 2-cyclohexen-1-yl, 3-cyclohexen-1-yl, 2,4-cyclohexadien-1-yl and 3-cyclopenten-1-yl.

The term “cycloalkyl” as used herein, means a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two adjacent or non-adjacent carbon atoms of the monocyclic ring are linked by an alkylene bridge of between one and three additional carbon atoms. Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantane).

The cycloalkyl groups of the invention are optionally substituted with 1, 2, 3, 4 or 5 substituents selected from the group consisting of alkenyl, alkoxy, alkoxyalkoxy, alkoxyalkyl, alkoxycarbonyl, alkoxysulfonyl, alkyl, alkylcarbonyl, alkylcarbonyloxy, alkylsulfonyl, alkylthio, alkylthioalkyl, alkynyl, carboxy, cyano, formyl, haloalkoxy, haloalkyl, halogen, hydroxy, hydroxyalkyl, mercapto, oxo, —NZ₁Z₂, and (NZ₃Z₄)carbonyl.

The term “cycloalkylalkyl” as used herein, means a cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl, and 4-cycloheptylbutyl.

The term “cycloalkylcarbonyl” as used herein, means cycloalkyl group, as defined herein, appended to the parent molecular moiety through a carbonyl group, as defined herein. Representative examples of cycloalkylcarbonyl include, but are not limited to, cyclopropylcarbonyl, 2-cyclobutylcarbonyl, and cyclohexylcarbonyl.

The term “cycloalkyloxy” as used herein, means cycloalkyl group, as defined herein, appended to the parent molecular moiety through an oxygen atom, as defined herein. Representative examples of cycloalkyloxy include, but are not limited to, cyclopropyloxy, cyclobutyloxy, cyclopentyloxy, cyclohexyloxy, cycloheptyloxy, and cyclooctyloxy.

The term “cycloalkylthio” as used herein, means cycloalkyl group, as defined herein, appended to the parent molecular moiety through a sulfur atom, as defined herein. Representative examples of cycloalkylthio include, but are not limited to, cyclopropylthio, cyclobutylthio, cyclopentylthio, cyclohexylthio, cycloheptylthio, and cyclooctylthio.

The term “ethylenedioxy” as used herein, means —O(CH₂)₂O— group wherein the oxygen atoms of the ethylenedioxy group are attached to the parent molecular moiety through one carbon atom forming a 5 membered ring or the oxygen atoms of the ethylenedioxy group are attached to the parent molecular moiety through two adjacent carbon atoms forming a six membered ring.

The term “formyl” as used herein, means a —C(O)H group.

The term “formylalkyl” as used herein, means a formyl group, as defined herein, appended to the parent molecular moiety through an alkyl group, as defined herein. Representative examples of formylalkyl include, but are not limited to, formylmethyl and 2-formylethyl.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means a monocyclic heteroaryl or a bicyclic heteroaryl. The monocyclic heteroaryl is a 5 or 6 membered ring that contains at least one heteroatom selected from the group consisting of nitrogen, oxygen and sulfur. The 5 membered ring contains two double bonds and the 6 membered ring contains three double bonds. The 5 or 6 membered heteroaryl is connected to the parent molecular moiety through any carbon atom or any substitutable nitrogen atom contained within the heteroaryl, provided that proper valance is maintained. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any substitutable nitrogen atom contained within the bicyclic heteroaryl, provided that proper valance is maintained. Representative examples of bicyclic heteroaryl include, but are not limited to, azaindolyl, benzimidazolyl, benzofuranyl, benzoxadiazolyl, benzoisoxazole, benzoisothiazole, benzooxazole, 1,3-benzothiazolyl, benzothienyl(or benzothiophenyl), cinnolinyl, furopyridine, indolyl, indazolyl, indolinonyl, isobenzofuran, isoindolyl, isoquinolinyl, naphthyridinyl, oxadiazolyl, oxazolopyridine, quinolinyl, quinoxalinyl, thiadiazolyl, and thienopyridinyl.

The heteroaryl groups of the invention are optionally substituted with 1, 2, 3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, nitro, —NZ₁Z₂ and (NZ₃Z₄)carbonyl. Heteroaryl groups of the invention that are substituted with a hydroxy group may be present as tautomers. The heteroaryl groups of the invention encompasses all tautomers including non-aromatic tautomers. In addition, the nitrogen heteroatoms can be optionally quaternized or oxidized to the N-oxide.

The term “heteroarylalkoxy” as used herein, means a heteroaryl group, as defined herein, appended to the parent molecular moiety through an alkoxy group, as defined herein. Representative examples of heteroarylalkoxy include, but are not limited to, fur-3-ylmethoxy, 1H-imidazol-2-ylmethoxy, 1H-imidazol-4-ylmethoxy, 1-(pyridin-4-yl)ethoxy, pyridin-3-ylmethoxy, 6-chloropyridin-3-ylmethoxy, pyridin-4-ylmethoxy, (6-(trifluoromethyl)pyridin-3-yl)methoxy, (6-(cyano)pyridin-3-yl)methoxy, (2-(cyano)pyridin-4-yl)methoxy, (5-(cyano)pyridin-2-yl)methoxy, (2-(chloro)pyridin-4-yl)methoxy, pyrimidin-5-ylmethoxy, 2-(pyrimidin-2-yl)propoxy, thien-2-ylmethoxy, and thien-3-ylmethoxy.

The term “heteroarylalkyl” as used herein, means a heteroaryl, as defined herein, appended to the parent molecular moiety through an alkyl group, as defined herein. Representative examples of heteroarylalkyl include, but are not limited to, fur-3-ylmethyl, 1H-imidazol-2-ylmethyl, 1H-imidazol-4-ylmethyl, 1-(pyridin-4-yl)ethyl, pyridin-3-ylmethyl, 6-chloropyridin-3-ylmethyl, pyridin-4-ylmethyl, (6-(trifluoromethyl)pyridin-3-yl)methyl, (6-(cyano)pyridin-3-yl)methyl, (2-(cyano)pyridin-4-yl)methyl, (5-(cyano)pyridin-2-yl)methyl, (2-(chloro)pyridin-4-yl)methyl, pyrimidin-5-ylmethyl, 2-(pyrimidin-2-yl)propyl, thien-2-ylmethyl, and thien-3-ylmethyl.

The term “heteroarylalkylcarbonyl” as used herein, means a heteroarylalkyl, as defined herein, appended to the parent molecular moiety through a carbonyl group, as defined herein.

The term “heteroarylalkylthio” as used herein, means a heteroarylalkyl group, as defined herein, appended to the parent molecular moiety through a sulfur atom. Representative examples of heteroarylalkylthio include, but are not limited to, fur-3-ylmethylthio, 1H-imidazol-2-ylmethylthio, 1H-imidazol-4-ylmethylthio, pyridin-3-ylmethylthio, 6-chloropyridin-3-ylmethylthio, pyridin-4-ylmethylthio, (6-(trifluoromethyl)pyridin-3-yl)methylthio, (6-(cyano)pyridin-3-yl)methylthio, (2-(cyano)pyridin-4-yl)methylthio, (5-(cyano)pyridin-2-yl)methylthio, (2-(chloro)pyridin-4-yl)methylthio, pyrimidin-5-ylmethylthio, 2-(pyrimidin-2-yl)propylthio, thien-2-ylmethylthio, and thien-3-ylmethylthio.

The term “heteroarylcarbonyl” as used herein, means a heteroaryl group, as defined herein, appended to the parent molecular moiety through a carbonyl group, as defined herein. Representative examples of heteroarylcarbonyl include, but are not limited to, fur-3-ylcarbonyl, 1H-imidazol-2-ylcarbonyl, 1H-imidazol-4-ylcarbonyl, pyridin-3-ylcarbonyl, 6-chloropyridin-3-ylcarbonyl, pyridin-4-ylcarbonyl, (6-(trifluoromethyl)pyridin-3-yl)carbonyl, (6-(cyano)pyridin-3-yl)carbonyl, (2-(cyano)pyridin-4-yl)carbonyl, (5-(cyano)pyridin-2-yl)carbonyl, (2-(chloro)pyridin-4-yl)carbonyl, pyrimidin-5-ylcarbonyl, pyrimidin-2-ylcarbonyl, thien-2-ylcarbonyl, and thien-3-ylcarbonyl.

The term “heteroaryloxy” as used herein, means a heteroaryl group, as defined herein, appended to the parent molecular moiety through an oxygen atom. Representative examples of heteroaryloxy include, but are not limited to, fur-3-yloxy, 1H-imidazol-2-yloxy, 1H-imidazol-4-yloxy, pyridin-3-yloxy, 6-chloropyridin-3-yloxy, pyridin-4-yloxy, (6-(trifluoromethyl)pyridin-3-yl)oxy, (6-(cyano)pyridin-3-yl)oxy, (2-(cyano)pyridin-4-yl)oxy, (5-(cyano)pyridin-2-yl)oxy, (2-(chloro)pyridin-4-yl)oxy, pyrimidin-5-yloxy, pyrimidin-2-yloxy, thien-2-yloxy, and thien-3-yloxy.

The term “heteroaryloxyalkyl” as used herein, means a heteroaryloxy group, as defined herein, appended to the parent molecular moiety through an alkyl group, as defined herein. Representative examples of heteroaryloxyalkyl include, but are not limited to, pyridin-3-yloxymethyl and 2-quinolin-3-yloxyethyl.

The term “heteroarylthio” as used herein, means a heteroaryl group, as defined herein, appended to the parent molecular moiety through a sulfur atom. Representative examples of heteroarylthio include, but are not limited to, pyridin-3-ylthio and quinolin-3-ylthio.

The term “heteroarylthioalkyl” as used herein, means a heteroarylthio group, as defined herein, appended to the parent molecular moiety through an alkyl group, as defined herein.

Representative examples of heteroarylthioalkyl include, but are not limited to, pyridin-3-ylthiomethyl, and 2-quinolin-3-ylthioethyl.

The term “heterocycle” or “heterocyclic” as used herein, means a monocyclic heterocycle, a bicyclic heterocycle or a tricyclic heterocycle. The monocyclic heterocycle is a 3, 4, 5, 6 or 7 membered ring containing at least one heteroatom independently selected from the group consisting of O, N, and S. The 3 or 4 membered ring contains 1 heteroatom selected from the group consisting of O, N and S. The 5 membered ring contains zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a 5 or 6 membered monocyclic heterocycle fused to a phenyl group, or a 5 or 6 membered monocyclic heterocycle fused to a cycloalkyl, or a 5 or 6 membered monocyclic heterocycle fused to a cycloalkenyl, or a 5 or 6 membered monocyclic heterocycle fused to a monocyclic heterocycle. The bicyclic heterocycle is connected to the parent molecular moiety through any carbon atom or any nitrogen atom contained within the bicyclic heterocycle. Representative examples of bicyclic heterocycle include, but are not limited to, 1,3-benzodioxolyl, 1,3-benzodithiolyl, 2,3-dihydro-1,4-benzodioxinyl, benzodioxolyl, 2,3-dihydro-1-benzofuranyl, 2,3-dihydro-1-benzothienyl, chromenyl and 1,2,3,4-tetrahydroquinolinyl. The tricyclic heterocycle is a bicyclic heterocycle fused to a phenyl, or a bicyclic heterocycle fused to a cycloalkyl, or a bicyclic heterocycle fused to a cycloalkenyl, or a bicyclic heterocycle fused to a monocyclic heterocycle. The tricyclic heterocycle is connected to the parent molecular moiety through any carbon atom or any nitrogen atom contained within the tricyclic heterocycle. Representative examples of tricyclic heterocycle include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl.

The heterocycles of this invention are optionally substituted with 1, 2, 3 or 4 substituents independently selected from the group consisting of alkenyl, alkoxy, alkoxyalkoxy, alkoxyalkyl, alkoxycarbonyl, alkoxycarbonylalkyl, alkoxysulfonyl, alkyl, alkylcarbonyl, alkylcarbonylalkyl, alkylcarbonyloxy, alkylthio, alkylthioalkyl, alkynyl, carboxy, carboxyalkyl, cyano, cyanoalkyl, formyl, haloalkoxy, haloalkyl, halogen, hydroxy, hydroxyalkyl, mercapto, oxo, —NZ₁Z₂ and (NZ₃Z₄)carbonyl.

The term “heterocyclealkoxy” as used herein, means a heterocycle group, as defined herein, appended to the parent molecular moiety through an alkoxy group, as defined herein. Representative examples of heterocyclealkoxy include, but are not limited to, 2-pyridin-3-ylethoxy, 3-quinolin-3-ylpropoxy, and 5-pyridin-4-ylpentyloxy.

The term “heterocyclealkyl” as used herein, means a heterocycle, as defined herein, appended to the parent molecular moiety through an alkyl group, as defined herein. Representative examples of heterocyclealkyl include, but are not limited to, piperidin-4-ylmethyl, piperazin-1-ylmethyl, 3-methyl-1-pyrrolidin-1-ylbutyl, (1R)-3-methyl-1-pyrrolidin-1-ylbutyl, (1S)-3-methyl-1-pyrrolidin-1-ylbutyl.

The term “heterocyclealkylcarbonyl” as used herein, means a heterocyclealkyl, as defined herein, appended to the parent molecular moiety through a carbonyl group, as defined herein. Representative examples of heterocyclealkylcarbonyl include, but are not limited to, piperidin-4-ylmethylcarbonyl, piperazin-1-ylmethylcarbonyl, 3-methyl-1-pyrrolidin-1-ylbutylcarbonyl, (1R)-3-methyl-1-pyrrolidin-1-ylbutylcarbonyl, (1S)-3-methyl-1-pyrrolidin-1-ylbutylcarbonyl.

The term “heterocyclealkylthio” as used herein, means a heterocyclealkyl group, as defined herein, appended to the parent molecular moiety through a sulfur atom. Representative examples of heterocyclealkylthio include, but are not limited to, 2-pyridin-3-ylethylthio, 3-quinolin-3-ylpropythio, and 5-pyridin-4-ylpentylthio.

The term “heterocyclecarbonyl” as used herein, means a heterocycle, as defined herein, appended to the parent molecular moiety through a carbonyl group, as defined herein.

The term “heterocyclecarbonylalkyl” as used herein, means a heterocyclecarbonyl, as defined herein, appended to the parent molecular moiety through an alkyl group, as defined herein.

The term “heterocycleoxy” as used herein, means a heterocycle group, as defined herein, appended to the parent molecular moiety through an oxygen atom. Representative examples of heterocycleoxy include, but are not limited to, pyridin-3-yloxy and quinolin-3-yloxy.

The term “heterocycleoxyalkyl” as used herein, means a heterocycleoxy group, as defined herein, appended to the parent molecular moiety through an alkyl group, as defined herein. Representative examples of heterocycleoxyalkyl include, but are not limited to, pyridin-3-yloxymethyl and 2-quinolin-3-yloxyethyl.

The term “heterocyclethio” as used herein, means a heterocycle group, as defined herein, appended to the parent molecular moiety through a sulfur atom. Representative examples of heterocyclethio include, but are not limited to, pyridin-3-ylthio and quinolin-3-ylthio.

The term “heterocyclethioalkyl” as used herein, means a heterocyclethio group, as defined herein, appended to the parent molecular moiety through an alkyl group, as defined herein. Representative examples of heterocyclethioalkyl include, but are not limited to, pyridin-3-ylthiomethyl, and 2-quinolin-3-ylthioethyl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “hydroxy-protecting group” or “O-protecting group” means a substituent which protects hydroxy groups against undesirable reactions during synthetic procedures. Examples of hydroxy-protecting groups include, but are not limited to, substituted methyl ethers, for example, methoxymethyl, benzyloxymethyl, 2-methoxyethoxymethyl, 2-(trimethylsilyl)-ethoxymethyl, benzyl, and triphenylmethyl; tetrahydropyranyl ethers; substituted ethyl ethers, for example, 2,2,2-trichloroethyl and t-butyl; silyl ethers, for example, trimethylsilyl, t-butyldimethylsilyl and t-butyldiphenylsilyl; cyclic acetals and ketals, for example, methylene acetal, acetonide and benzylidene acetal; cyclic ortho esters, for example, methoxymethylene; cyclic carbonates; and cyclic boronates. Commonly used hydroxy-protecting groups are disclosed in T. W. Greene and P. G. M. Wuts, Protective Groups in Organic Synthesis, 3rd edition, John Wiley & Sons, New York (1999).

The term “lower alkenyl” as used herein, is a subset of alkenyl, as defined herein, and means an alkenyl group containing from 2 to 4 carbon atoms. Examples of lower alkenyl are ethenyl, propenyl, and butenyl.

The term “lower alkoxy” as used herein, is a subset of alkoxy, as defined herein, and means a lower alkyl group, as defined herein, appended to the parent molecular moiety through an oxygen atom, as defined herein. Representative examples of lower alkoxy include, but are not limited to, methoxy, ethoxy, propoxy, 2-propoxy, butoxy, and tert-butoxy.

The term “lower alkyl” as used herein, is a subset of alkyl as defined herein and means a straight or branched chain hydrocarbon group containing from 1 to 4 carbon atoms. Examples of lower alkyl are methyl, ethyl, n-propyl, iso-propyl, n-butyl, iso-butyl, sec-butyl, and tert-butyl.

The term “lower alkylthio” as used herein, is a subset of alkylthio, means a lower alkyl group, as defined herein, appended to the parent molecular moiety through a sulfur atom. Representative examples of lower alkylthio include, but are not limited to, methylthio, ethylthio, and tert-butylthio.

The term “lower alkynyl” as used herein, is a subset of alkynyl, as defined herein, and means an alkynyl group containing from 2 to 4 carbon atoms. Examples of lower alkynyl are ethynyl, propynyl, and butynyl.

The term “lower haloalkoxy” as used herein, is a subset of haloalkoxy, as defined herein, and means a straight or branched chain haloalkoxy group containing from 1 to 4 carbon atoms. Representative examples of lower haloalkoxy include, but are not limited to, trifluoromethoxy, trichloromethoxy, dichloromethoxy, fluoromethoxy, and pentafluoroethoxy.

The term “lower haloalkyl” as used herein, is a subset of haloalkyl, as defined herein, and means a straight or branched chain haloalkyl group containing from 1 to 4 carbon atoms. Representative examples of lower haloalkyl include, but are not limited to, trifluoromethyl, trichloromethyl, dichloromethyl, fluoromethyl, and pentafluoroethyl.

The term “mercapto” as used herein, means a —SH group.

The term “mercaptoalkyl” as used herein, means a mercapto group, as defined herein, appended to the parent molecular moiety through an alkyl group, as defined herein. Representative examples of mercaptoalkyl include, but are not limited to, 2-mercaptoethyl and 3-mercaptopropyl.

The term “methylenedioxy” as used herein, means a —OCH₂O— group wherein the oxygen atoms of the methylenedioxy are attached to the parent molecular moiety through two adjacent carbon atoms.

The term “nitrogen protecting group” as used herein, means those groups intended to protect an amino group against undesirable reactions during synthetic procedures. Preferred nitrogen protecting groups are acetyl, benzoyl, benzyl, benzyloxycarbonyl (Cbz), formyl, phenylsulfonyl, tert-butoxycarbonyl (Boc), tert-butylacetyl, trifluoroacetyl, and triphenylmethyl (trityl).

The term “nitro” as used herein, means a —NO₂ group.

The term “NZ₁Z₂” as used herein, means two groups, Z₁ and Z₂, which are appended to the parent molecular moiety through a nitrogen atom. Z₁ and Z₂ are each independently selected from the group consisting of hydrogen, alkyl, alkylcarbonyl, alkoxycarbonyl, aryl, arylalkyl, formyl and (NZ₅Z₆)carbonyl. In certain instances within the invention, Z₁ and Z₂ taken together with the nitrogen atom to which they are attached form a heterocyclic ring. Representative examples of NZ₁Z₂ include, but are not limited to, amino, methylamino, acetylamino, acetylmethylamino, phenylamino, benzylamino, azetidinyl, pyrrolidinyl and piperidinyl.

The term “NZ₃Z₄” as used herein, means two groups, Z₃ and Z₄, which are appended to the parent molecular moiety through a nitrogen atom. Z₃ and Z₄ are each independently selected from the group consisting of hydrogen, alkyl, aryl and arylalkyl. Representative examples of NZ₃Z₄ include, but are not limited to, amino, methylamino, phenylamino and benzylamino.

The term “NZ₅Z₆” as used herein, means two groups, Z₅ and Z₆, which are appended to the parent molecular moiety through a nitrogen atom. Z₅ and Z₆ are each independently selected from the group consisting of hydrogen, alkyl, aryl and arylalkyl. Representative examples of NZ₅Z₆ include, but are not limited to, amino, methylamino, phenylamino and benzylamino.

The term “(NZ₃Z₄)carbonyl” as used herein, means a NZ₃Z₄ group, as defined herein, appended to the parent molecular moiety through a carbonyl group, as defined herein. Representative examples of (NZ₃Z₄)carbonyl include, but are not limited to, aminocarbonyl, (methylamino)carbonyl, (dimethylamino)carbonyl, and (ethylmethylamino)carbonyl.

The term “oxo” as used herein, means a ═O moiety.

The term “sulfinyl” as used herein, means a —S(O)— group.

The term “sulfonyl” as used herein, means a —SO₂— group.

The term “tautomer” as used herein means a proton shift from one atom of a compound to another atom of the same compound wherein two or more structurally distinct compounds are in equilibrium with each other.

The term “linkage disequilibrium” describes a situation in which some combinations of alleles or genetic markers occur more or less frequently in a population than would be expected from a random formation of haplotypes from alleles based on their frequencies. When a particular allele at one locus is found together on the same chromosome with a specific allele at a second locus-more often than expected if the loci were segregating independently in a population—the loci are in disequilibrium.

The term “pharmaceutically suitable excipient” refers to a solid, semi-solid or liquid fillers, diluents, encapsulating material, formulation auxiliary suitable for administering to a subject. Examples of pharmaceutically suitable excipients include, but are not limited to, sugars, cellulose and derivatives thereof, oils, glycols, solutions, buffers, colorants, releasing agents, coating agents, sweetening agents, flavoring agents, perfuming agents, and the like. Such therapeutic compositions may be administered parenterally, intracisternally, orally, rectally, intraperitoneally or by other dosage forms known in the art.

The term “therapeutically suitable metabolite” refers to a pharmaceutically active compound formed by the in vivo biotransformation of compounds of formula (I-V).

The term “therapeutically suitable prodrug,” refers to those prodrugs or zwitterions which are suitable for use in contact with the tissues of patients without undue toxicity, irritation, and allergic response, are commensurate with a reasonable benefit/risk ratio, and are effective for their intended use. The term “prodrug,” refers to compounds that are rapidly transformed in vivo to the compounds of formula (I-V) for example, by hydrolysis in blood.

The term “prodrug,” refers to compounds that contain, but are not limited to, substituents known as “therapeutically suitable esters.” The term “therapeutically suitable ester,” refers to alkoxycarbonyl groups appended to the parent molecule on an available carbon atom. More specifically, a “therapeutically suitable ester,” refers to alkoxycarbonyl groups appended to the parent molecule on one or more available aryl, cycloalkyl and/or heterocycle groups as defined herein. Compounds containing therapeutically suitable esters are an example, but are not intended to limit the scope of compounds considered to be prodrugs. Examples of prodrug ester groups include pivaloyloxymethyl, acetoxymethyl, phthalidyl, indanyl and methoxymethyl, as well as other such groups known in the art. Other examples of prodrug ester groups are found in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987.

The term “smoker” refers to a person or patient that smokes more than 1, 2, 3, 4, 5, 6, 7, 8, 9, 10, 11, 12, 13, 14, 15, 16, 17, 18, 19, 20 or more cigarettes a day, i.e., a regular basis. A patient classified as a smoker may be a person who smokes more than ½, 1, 1 and ½, or 2 packs a day.

A “non-smoker” or a “nonsmoking patient” is a person or patient who has not smoked on a regular basis for at least 3 months prior to the initial screening conducted during the clinical study. A nonsmoking patient may have a negative cotinine test result during the screening procedures. As such it is recognized that nonsmoking patients are those who have not engaged in smoking on a regular basis for a significant number of days, for example at least 90 days.

The terms “subject” and “patient” are used interchangeably irrespective of whether the subject has or is currently undergoing any form of treatment.

The terms “weight percent” or “percent by weight” or “% by weight” or “wt %” denote the weight of an individual component in a composition or mixture as a percentage of the weight of the composition or mixture.

Substituents attached to a cyclic moiety, for instance a cycloalkyl, aryl, or heterocycloalkyl moiety, can be represented as not bound to any particular atom, but rather as attached to bonds that perpendicularly intersect a side of the cyclic group. This notation is meant to indicate that the substituent can be bound to one of two or more atoms of the cyclic group.

Although typically it may be recognized that an asterisk is used to indicate that the exact subunit composition of a receptor is uncertain, for example a3b4* indicates a receptor that contains the a3 and b4 proteins in combination with other subunits, the term α7 as used herein is intended to include receptors wherein the exact subunit composition is both certain and uncertain. For example, as used herein α7 includes homomeric (α7)5 receptors and α7* receptors, which denote a nAChR containing at least one α7 subunit.

### 2. Method of Improving Cognition Impairment of Patients Suffering from Schizophrenia or Related Schizophrenia Psychotic Disorders

The present invention is directed to methods for improving cognition impairment associated with a patient suffering from schizophrenia, schizophreniform disorder or a related schizophrenia spectrum psychotic disorder by correlating genetic variation in the catechol-O-methyltransferase (COMT) gene with particular nicotinic acetylcholine receptors (nAChRs) ligand modulators. The method's correlation may be affected by whether the patient is a smoker or a non-smoker.

The method may further be directed to the steps of (1) obtaining a sample from the patient; (2) determining the identity of an allele of at least one single nucleotide polymorphism (SNP) locus in the COMT gene in the sample; (3) determining the smoking status of the patient; (4) identifying the patient as a candidate for effective treatment with a particular nAChR ligand based upon the presence or absence of a particular SNP allele in the COMT gene in the sample and the smoking status of the patient; (5) administering an effective dosage of the particular nAChR ligand modulator to the patient identified as being a candidate for effective treatment for improving cognition impairment; and (6) measuring the cognitive ability of the patient. The nAChR ligand is described below.

The method takes advantage of nicotinic receptors ability to mediate a very wide range of physiological effects, and have been targeted for therapeutic treatment of disorders relating to cognitive function, learning and memory, neurodegeneration, pain, inflammation, psychosis, sensory gating, mood, and emotion, among other conditions. Many subtypes of the nAChR exist in the CNS and periphery. Each subtype has a different effect on regulating the overall physiological function. Typically, nAChRs are ion channels that are constructed from a pentameric assembly of subunit proteins. At least 12 subunit proteins, α2-α10 and β2-β4, have been identified in neuronal tissue. These subunits provide for a great variety of homomeric and heteromeric combinations that account for the diverse receptor subtypes. For example, the predominant receptor that is responsible for high affinity binding of nicotine in brain tissue has composition (α4) β(2)3 (the α4 β2 subtype), while another major population of receptors is comprised of homomeric (α7)5 (the α7 subtype) receptors.

The method includes collecting samples (also referred to as “specimens”) from a patient suffering from schizophrenia, schizophreniform disorder or a related schizophrenia spectrum psychotic disorder. The method can use a patient tissue sample of any type or on a derivative thereof, including peripheral blood, serum or plasma fraction from peripheral blood, tumor or suspected tumor tissues (including fresh frozen and fixed or paraffin embedded tissue), cell isolates such as circulating epithelial cells separated or identified in a blood sample, lymph node tissue, bone marrow and fine needle aspirates. The sample suitable for use in the method can comprise any tissue type or cell isolates from any tissue type, including a peripheral blood sample, a tumor tissue or a suspected tumor tissue, a thin layer cytological sample, a fine needle aspirate sample, a bone marrow sample, a lymph node sample, a urine sample, a saliva sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample, a paraffin embedded tissue sample or an extract or processed sample produced from any of a peripheral blood sample, a serum or plasma fraction of a peripheral blood sample, a tumor tissue or a suspected tumor tissue, a thin layer cytological sample, a fine needle aspirate sample, a bone marrow sample, a lymph node sample, a urine sample, a saliva sample, an ascites sample, a lavage sample, an esophageal brushing sample, a bladder or lung wash sample, a spinal fluid sample, a brain fluid sample, a ductal aspirate sample, a nipple discharge sample, a pleural effusion sample, a fresh frozen tissue sample or a paraffin embedded tissue sample. For example, a patient peripheral blood sample can be initially processed to extract an epithelial cell population, a plasma fraction or a serum fraction, and this extract, plasma fraction or serum fraction can then be assayed. A microdissection of the tissue sample to obtain a cellular sample enriched with suspected tumor cells can also be used.

From any of the above described samples, genomic DNA can be isolated. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA. The genomic DNA sample can then treated with a reagent in such manner (such as by using a bisulfite reagent) that cytosine bases which are unmethylated at the 5′-position are converted to uracil, thymine, or another base which is dissimilar to cytosine in terms of hybridization behavior.

a. Correlation of COMT with nAChR

The method takes advantage of the affect of particular genetic variations of the COMT gene and modulation of nAChR. COMT is one of the major mammalian enzymes involved in metabolic degradation of catecholamines. COMT catalyzes the transfer of a methyl group from S-adenosyl-methionine to catecholamines, including the neurotransmitters dopamine, epinephrine and norepinephrine. This results in one of the major degradative pathways of the catecholamine transmitters.

The COMT gene consists of 8 exons and is localized to chromosome 22q11.2 in humans. A number of genetic variants in the COMT gene have been identified. The most studied variation is a single base pair substitution of guanine for adenine, which results in the replacement of valine with methionine at position 158 (rs4680). Studies have shown that the substitution of methionine lowers the enzymes thermostability, resulting in a reduction on COMT activity. This variant has been associated in a number of studies with etiology of disease disorders or with response to new drug therapies, including nicotine addiction, schizophrenia, and treatment response to major depressive disorders. Other variants that have been extensively studied for association to disease states include rs4818, rs4633 and rs6269.

The genetic variants of COMT may be any single genetic polymorphism of the COMT gene.

The genetic variants of COMT may be different from a reference COMT sequence. The genetic variant may be one or more SNPs isolated from the human COMT gene variant sequences NM_{—}00754.3, NM_{—}001135162.1, NM_{—}001135161.1, NM_{—}007310.2, and NM_{—}00796.3 as indicated in the National Center for Biotechnology Information. The genetic variant may be any SNP associated with the COMT gene, such as any SNP in the COMT gene or any SNP in the region surrounding the COMT gene.

The genetic variants of COMT may be a polymorphic site associated with at least one SNP of rs4818, rs4680, rs4633 and rs6269, or a SNP in linkage disequilibrium with at least one of the foregoing SNPs, or combinations thereof. The genetic variants of COMT may be a polymorphic site associated with at least one SNP of rs4818, rs4680, rs4633 and rs6269, or combinations thereof. The genetic variants of COMT may be a polymorphic site being in complete or strong linkage disequilibrium with at least one SNP of rs4818, rs4680, rs4633 and rs6269, or combinations thereof.

The genetic variants of COMT may be rs4818 of NCBI wherein C/C is the reference or major allele, and G/C or G/G is the SNPs with G being the minor allele. The genetic variant of COMT may be rs4680 wherein G/G is the reference or major allele, and A/A or G/A is the SNPs with A being the minor allele. The genetic variant of COMT may be rs4633 wherein C/C is the reference or major allele, and T/T or C/T is the SNP reference with T being the minor allele. The genetic variant may be rs6269 wherein A/A is the reference or major allele, and G/G or A/G is the SNP with G being the minor allele.

The method may also identify genetic variants of COMT that can be correlated with a particular NaChR modulator. The method may identify and determine patterns of genetic variants of COMT that can be correlated with a particular NaChR modulator, i.e., the presence or absence of a particular genetic variant may correlate with responsiveness to a particular NaChR modulator. For example, if the patient is a non-smokers and has the minor allele for rs6269 and rs4818, the patient may be more responsive to NaChR modulator treatment than the non-smokers who had the major allele at these loci. If the patient is a non-smoker and has the major allele for rs4633 and rs4680, this patient may be more responsive to nAChR modulator treatment than the non-smokers who had the minor allele at these loci.

In another embodiment of the invention, if the patient is a non-smoker and has the minor allele for rs6269 and rs4818, this patient may be more responsive to treatment with the nAChR agonist (4s)-4-(5-phenyl-1,3,4-thiadiazol-2-yloxy)-1-azatricyclo[3.3.1.1^{3,7}]decane (Compound A) (as discussed below) than the non-smokers who had the major allele at these loci. If the patient is a non-smoker and has the major allele for rs4633 and rs4680, this patient may be more responsive to treatment with the nAChR agonist (4s)-4-(5-phenyl-1,3,4-thiadiazol-2-yloxy)-1-azatricyclo[3.3.1.1^{3,7}]decane (Compound A) (as discussed below) than the non-smokers who had the minor allele at these loci.

The method may also identify additional genetic variants associated with the COMT gene, for example, the method may identify additional SNPs associated with the COMT gene which may demonstrate a similar relationship with a particular NaChR modulator. Other SNPs associated with the COMT gene, such as SNPs in the COMT gene or regions surrounding the COMT gene may be identified to determine if there are other SNPs that could be used to identify patients for effective treatment with a nAChR modulator. The method may include further correlating combinations of SNPs of COMT as good indicators that the patient will be responsive to nAChR modulator treatment.

b. Effect of Smoking on Genetic COMT Correlation and Use of a Particular NaChR Modulator

The method further comprises the step of determining the smoking status of the patient. The smoking status of the patient may affect the ability to correlate the genetic variant of COMT with a particular NaChR modulator. A patient who is a smoker may attenuate the responsiveness of nAChR modulator treatment if the patient has the major alleles at rs6269, rs4818, rs4633 and rs4680 of COMT. If a patient is a smoker and has minor alleles for rs6269, rs4818, rs4633 and rs4680, this patient may be more responsive to nAChR modulator treatment compared to smokers who had the major alleles. Accordingly, if a patient has the major allele and smokes, then treatment with an nAChR modulator would not be effective. However, a smoker with the minor allele would still see benefit from to nAChR modulator treatment.

Similarly, a patient who is a smoker may attenuate the responsiveness of treatment with nAChR agonist (4s)-4-(5-phenyl-1,3,4-thiadiazol-2-yloxy)-1-azatricyclo[3.3.1.1^{3,7}]decane (Compound A) (as discussed below) if the patient has the major alleles at rs6269, rs4818, rs4633 and rs4680 of COMT. If a patient is a smoker and has minor alleles for rs6269, rs4818, rs4633 and rs4680, this patient may be more responsive to treatment with the nAChR agonist (4s)-4-(5-phenyl-1,3,4-thiadiazol-2-yloxy)-1-azatricyclo[3.3.1.1^{3,7}]decane (Compound A) (as discussed below) compared to smokers who had the major alleles.

### 3. Method of Monitoring the Treatment of a Patient

The invention may also be directed to a method for monitoring the treatment of a patient. The method comprises (1) obtaining a sample from a patient wherein the patient is suffering from schizophrenia, schizophreniform disorder or a related schizophrenia spectrum psychotic disorder, and is already under a treatment regimen with a particular nAChr ligand modulator; (2) determining the identity of an allele of at least one single nucleotide polymorphism (SNP) locus in the COMT gene in the sample; (3) determining the smoking status of the patient; and if necessary, modifying the course of treatment including administering to the patient in need thereof a different nAChr ligand modulator based upon the presence or absence of particular SNPs of the patient's COMT gene. The method provides clinicians the ability to identify the most effective nAChr ligand modulator based upon the SNP profile of the patient's COMT gene. Again, depending on whether the patient is a smoker or a non-smoker will further modify the course of treatment.

### 4. Method of Identifying a Patient for a Particular nAChR Ligand Modulator Treatment

The invention may also be directed to a method of identifying a patient suffering from schizophrenia, schizophreniform disorder or a related schizophrenia spectrum psychotic disorder as a candidate for effective treatment with a nicotinic acetylcholine receptor ligand modulator. The method may comprise (1) obtaining a sample from the patient; (2) determining the identity of an allele of at least one single nucleotide polymorphism (SNP) locus in the catechol-O-methyltransferase (COMT) gene in the sample; (3) determining the smoking status of the patient with schizophrenia; (4) identifying the patient as a candidate for effective treatment with the nicotinic acetylcholine receptor ligand modulator based on the presence or absence of a particular SNP allele in the COMT gene in the sample and the smoking status of the patient with schizophrenia; and (5) administering to the patient in need thereof an effective amount of a particular nicotinic acetylcholine receptor ligand modulator based upon result of step (4). The method provides clinicians the ability the identify the most effective nAChr ligand modulator based upon the SNP profile of the patient's COMT gene. Again, depending on whether the patient is a smoker or a non-smoker will further modify the course of treatment.

### 5. Method of Identifying a Desirable Patient for nAChR Ligand Modulator Treatment

The invention may also be directed to a method of identifying a patient suffering from schizophrenia, schizophreniform disorder or a related schizophrenia spectrum psychotic disorder with an increased likelihood of response to treatment with a nAChR ligand modulator treatment. The method comprises (a) obtaining a sample from the patient; (b) determining the identity of an allele of at least one single nucleotide polymorphism (SNP) locus in the catechol-β-methyltransferase (COMT) gene in the sample; (c) determining the smoking status of the patient; and (d) identifying the patient as having an increased likelihood of response to treatment with the nAChR ligand modulator based on the presence or absence of a particular SNP allele in the COMT gene in the sample and the smoking status of the patient. The presence of at least one SNP allele in the COMT gene in the patient identifies the patient as a candidate for effective treatment with the nAChR ligand modulator. The method provides clinicians with the ability to identify the best candidate patients for nAChr ligand modulator therapy based upon the SNP profile of the patient's COMT gene. Again, depending on whether the patient is a smoker or a non-smoker will further modify the course of treatment.

### 6. Patients to be Treated by Method

The patients to be treated by the methods described above may be a patient schizophrenia, schizophreniform disorder or a related schizophrenia spectrum psychotic disorder. A schizophrenia spectrum psychotic disorder may be include, but are not limited to, schizotypal personality disorder, brief psychotic disorder, delusional disorder, and substance-induced psychotic disorder. Schizophrenia, schizophreniform, schizoaffective disorder, schizotypal personality disorder, brief psychotic disorder delusional disorder, and substance-induced psychotic disorder are collectively referred to as schizophrenia spectrum psychotic disorders.

The patient may further suffer from schizophreniform disorders. Schizophreniform disorder shares common symptoms with schizophrenia, however, the patient may demonstrate a shorter duration of disruptive symptoms and the patient's level of functioning may be less affected than a patient diagnosed with schizophrenia. Schizoaffective disorder has features of schizophrenia and an affective (or mood) disorder.

The nAChR ligand modulators or compositions comprising nAChR ligand modulators as described below are administered to a patient in need of schizophrenia therapy or antipsychotic treatment. Such patient generally has received a diagnosis of schizophrenia. Any therapeutically effective nAChR ligand modulator can be administered to patients who are clinically stable and receiving a current regimen of a typical antipsychotic medications. Use in patients who have not yet received atypical antipsychotic medication or patients no longer receiving atypical antipsychotic medication also is also contemplated

### 7. Nicotinic Acetylcholine Receptors (nAChRs) Ligand Modulators

The methods of the invention described above correlate particular SNP profiles of the COMT gene with particular nAChR ligand modulators. The nAChR ligand modulators may be an agonist or antagonist of the nicotinic acetylcholine receptors. The nAChR ligand modulator may target the nicotinic acetylcholine receptor is α-7 nicotinic receptor

The nAChR ligand may be an agonist. The nAChR ligand agonist may be a compound of the Formula (I),

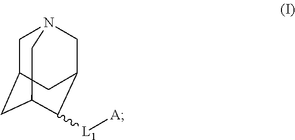

or a pharmaceutically acceptable salt or prodrug thereof, wherein

L₁ is —O— or —NRₐ—;

A is -Ar₁, -Ar₂-L₂-Ar₃ or -Ar₄-L₃-Ar₅;

Ar₁ is aryl or heteroaryl;

Ar₂ is aryl or monocyclic heteroaryl;

Ar₃ is aryl or heteroaryl;

Ar₄ is a bicyclic heteroaryl;

Ar₅ is aryl or heteroaryl;

L₂ is a bond, —O—, —NRₐ—, —CH₂—, or —C(O)NRₐ—;

L₃ is a bond, —O—, —NRₐ— or —CH₂—; and

Rₐ is hydrogen or alkyl.

Another embodiment is a nAChR agonist compound of formula (H),

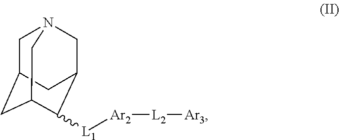

or a therapeutically suitable salt or prodrug thereof, wherein

Ar₂ is selected from

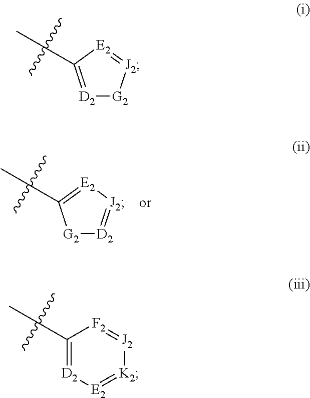

D₂, E₂, F₂, J₂, and K₂ are each independently —CT₂ or N;

G₂ is O, —NR₂ₐ; or S;

in each group of (i), (ii), and (iii), one substituent represented by T₂, or R₂ₐ wherein R₂ₐ is T₂, is -L₂-Ar₃ and the other substituents represented by T₂ are hydrogen, alkyl, alkoxy, alkoxycarbonyl, cyano, halo, nitro, or —NR_{b}R_{c};

R₂ₐ is hydrogen, alkyl, or T₂; and

R_{b} and R_{c}, are each independently hydrogen, alkyl, alkoxycarbonyl or alkylcarbonyl.

Ar₃ is a group selected from

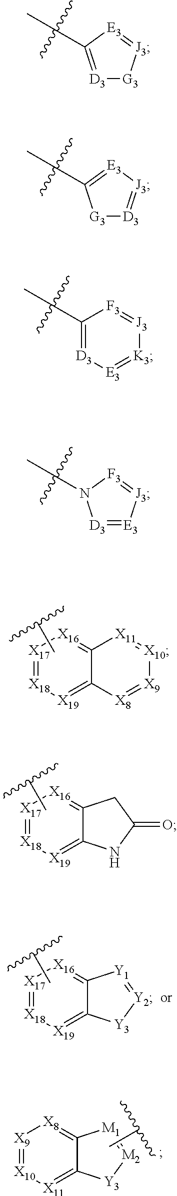

wherein D₃, E₃, F₃, J₃, K₃, X₈, X₉, X₁₀, and X₁₁ are each independently —CR₃ or N;

X₁₆, X₁₇, X₁₈, X₁₉, M₁, and M₂ are each independently —CR₃, N, or C;

G₃ is O; —NR₃ₐ; or S;

Y₁ is —CR₃ or N;

Y₂ is —CR₃ or N;

Y₃ is NH, O, or S;

R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano;

R₃ₐ is hydrogen, alkyl, alkylcarbonyl, tritylaryl, wherein aryl is preferably phenyl;

Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl;

one of X₁₆, X₁₇, X₁₈, and X₁₉, is C;

M₁ or M₂ is C;

L₁ is —O— or —NRₐ—;

L₂ is a bond, —O—, —NRₐ—, —CH₂—, or —C(O)NRₐ—; and

Rₐ is hydrogen or alkyl.

Another embodiment is a nAChR agonist compound of formula (III),

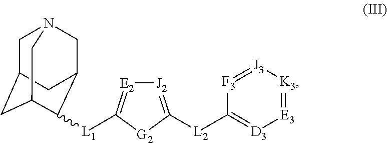

or a therapeutically suitable salt or prodrug thereof, wherein

E₂ and J₂ are each independently —CT₂ or N;

G₂ is O, —NR₂ₐ; or S;

T₂, at each occurrence, is independently hydrogen, alkyl, alkoxy, alkoxycarbonyl, cyano, halo, nitro, or —NR_{b}R_{c};

R₂ₐ is hydrogen, alkyl, or T₂;

R_{b} and R_{c} are each independently hydrogen, alkyl, alkoxycarbonyl or alkylcarbonyl;

D₃, E₃, F₃, J₃, and K₃ are each independently —CR₃ or N;

R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano;

Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl;

L₁ is —O— or —NRₐ—;

L₂ is a bond, —O—, —NRₐ—, —CH₂—, or —C(O)NRₐ—; and

Rₐ is hydrogen or alkyl.

Another embodiment is a compound of formula (IV),

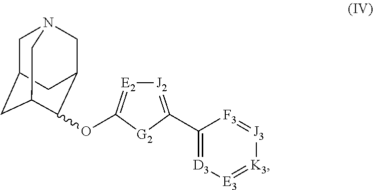

or a therapeutically suitable salt or prodrug thereof, wherein

E₂ and J₂ are each independently —CT₂ or N;

G₂ is O; —NR₂ₐ; or S;

T₂, at each occurrence, is independently hydrogen, alkyl, alkoxy, alkoxycarbonyl, cyano, halo, nitro, or —NR_{b}R_{c};

R₂ₐ is hydrogen, alkyl, or T₂;

R_{b} and R_{c} are each independently hydrogen, alkyl, alkoxycarbonyl or alkylcarbonyl;

D₃, E₃, F₃, J₃, and K₃ are each independently —CR₃ or N;

R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano; and

Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl.

Another embodiment is a compound of formula (V),

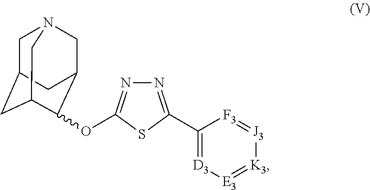

or a therapeutically suitable salt or prodrug thereof, wherein

D₃, E₃, F₃, J₃, and K₃ are each independently —CR₃ or N;

R₃ is hydrogen, alkyl, alkoxy, alkoxylalkyl, alkoxycarbonyl, alkylcarbonyl, cyano, halo, haloalkoxy, haloalkyl, hydroxy, nitro, RₑR_{f}N—, or aryl, wherein aryl is preferably phenyl optionally substituted with halo, alkyl or cyano; and

Rₑ and R_{f} are each independently hydrogen, alkyl, alkoxycarbonyl, or alkylcarbonyl, or Rₑ and R_{f} are each taken together with the nitrogen atom to which they are attached form a heterocyclic ring, wherein the heterocyclic ring is preferably pyrrolidinyl, piperidinyl or piperazinyl. The preparation of nAChR modulators of the invention are disclosed US Patent Application Publication No. 20080167336.

The nAChR ligand agonist may be a compound of the formula (VI),

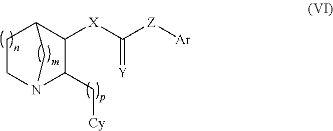

where in formula (VI)

m is 2;

n is 1;

p is 1, 2, 3 or 4;

X is oxygen or NR′;

Y is oxygen or sulfur;

Z is NR′, a covalent bond or a linker species A;

A is selected from the group —CR′R″—, —CR′R″—CR′R″—, —CR′═CR′— and —C═C—;

when Z is a covalent bond or A, X must be nitrogen;

Ar is an unsubstituted or substituted, carbocyclic or heterocyclic, monocyclic or fused polycyclic aryl group;

Cy is an unsubstituted or substituted 5- or 6-membered heteroaromatic ring; and substituents are selected from the group consisting of alkyl, alkenyl, heterocyclyl, cycloalkyl, aryl, substituted aryl, arylalkyl, substituted arylalkyl, halo, —OR′, —NR′R″, —CF3, —CN, —NO2, —R′, —SR′, —N3, —C(═O)NR′R″, —NR′C(═O)R″, —C(═O)R′, —C(═O)OR′, —OC(═O)R′, —O(CR′R″)rC(═O)R′, —O(CR′R″)rNR″C(═O)R′, —O(CR′R″)—NR″SO2R′, —OC(═O)NR′R″, —NR′C(═O)OR″, —SO2R′, —SO2NR′R″, and —NR′SO2R″;

wherein each of R′ and R″ individually is hydrogen, C1-C8 alkyl, C3-C8 cycloalkyl, heterocyclyl, aryl, or arylalkyl; or R′ and R″ can combine to form a 3 to 8 membered ring; and r is 1, 2, 3, 4, 5, or 6, or a pharmaceutically acceptable salt thereof.

Another compound which may be used for the methods may be TC-5619 (N-[2-(pyridin-3-ylmethyl)-1-azabicyclo[2.2.2]oct-3-yl]-1-benzofuran-2-carboxamide), which has been disclosed to be a neuronal nicotinic receptor agonist selective for α7 subtype.

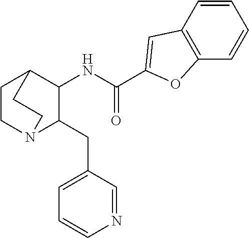 [SMILES: O=C(NC1C2CCN(CC2)C1Cc1cccnc1)c1cc2ccccc2o1]

The preparation of TC-5619 (N-[2-(pyridin-3-ylmethyl)-1-azabicyclo[2.2.2]oct-3-yl]-1-benzofuran-2-carboxamide) is disclosed U.S. Pat. No. 6,953,855.

The nAChR ligand agonist may be a compound of the Formula (VII),

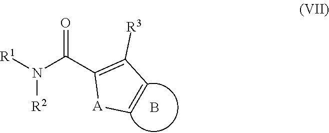

wherein in formula (VII)

R¹ represents 1-azabicyclo[2.2.2]oct-3-yl,

R² represents hydrogen or C₁-C₆-alkyl,

R³ represents hydrogen, halohalogen or C₁-C₆-alkyl,

A represents oxygen or sulfur, and

the ring B represents benzo, pyrimido, pyrimidazo or pyridazino which is substituted by a radical selected from the group consisting of halogen, C₁-C₆-alkanoyl, carbamoyl, cyano, trifluoromethyl, trifluoromethoxy, nitro, amino, C₁-C₆-acylamino, C₁-C₆-alkyl, C₁-C₆-alkyoxy, C₁-C₆-alkylthio, C₁-C₆-alkylamino, heteroarylcarbonylamino, arylcarbonylamino, C₁-C₆-alkylsulfonyl-amino, di(C₁-C₄-alkylsulfonyl)amino, arylsulfonylamino, di(arylsulfonyl)amino, C₃-C₆-cycloalkylcarbonylmethyl, 1,3-dioxa-propane-1,3-diyl, amino(hydroxyimino)methyl and benzo, or a salt, a hydrate or a hydrate of a salt thereof.

The nAChR ligand agonist may be a compound of the formula (VIII),

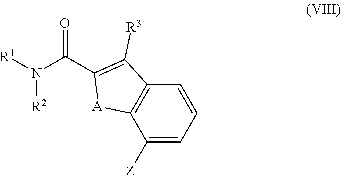

wherein in formula (VIII)

R¹ represents 1-azabicyclo[2.2.2]oct-3-yl,

R² represents hydrogen or C₁-C₆-alkyl,

R³ represents hydrogen, halogen or C₁-C₆-alkyl,

A represents oxygen or sulfur,

and Z represents halogen, formyl, carbamoyl, cyano, trifluoromethyl, trifluoromethoxy, nitro, amino, formamido, acetamido, C₁-C₆-alkyl, C₁-C₆-alkyoxy, C₁-C₆-alkylthio, C₁-C₆-alkylamino, heteroaryl-carbonylamino, arylcarbonylamino, C₁-C₄-alkylsulfonylamino, di(arylsulfonyl)amino, C₃-C₆-cycloalkylcarbonylmethyl or amino(hydroxyimino)methyl, or a salt, a hydrate or a hydrate of a salt thereof.

Another compound which may be used for the methods may be EVP-6124, which has been disclosed to be a neuronal nicotinic receptor partial agonist selective for α7 subtype. The preparation of EVP-6124 (N-[(3R)-1-azabicyclo[2.2.2]oct-3-yl]-7-chloro-1-benzothiophene-2-carboxamide) is disclosed in U.S. Pat. No. 7,732,477.

The nAChR ligand agonist may be (R)-7-chloro-N-(quinuclidin-3-yl)benzo[b]thiophene-2-carboxamide and has the following structure:

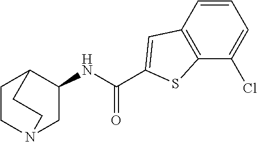 [SMILES: O=C(N[C@H]1CN2CCC1CC2)c1cc2cccc(Cl)c2s1]

### a. (4s)-4-(5-phenyl-1,3,4-thiadiazol-2-yloxy)-1-azatricyclo[3.3.1.1^{3,7}]decane (Compound A)

The methods of the invention described above correlate particular SNP profiles of the COMT gene with particular nAChR ligand agonist. The nAChR ligand agonist may be (4s)-4-(5-phenyl-1,3,4-thiadiazol-2-yloxy)-1-azatricyclo[3.3.1.1^{3,7}]decane (ABT-126 or Compound A) and has the following structure:

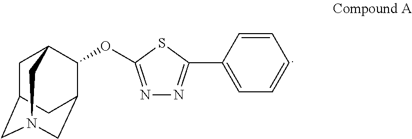 [SMILES: c1ccc(-c2nnc(O[C@@H]3C4CC5C[C@H]3C[N@](C5)C4)s2)cc1]

Alternatively, Compound A may also be called (1R,4R,5S)-4-(5-phenyl-1,3,4-thiadiazol-2-yloxy)-1-azatricyclo[3.3.1.1^{3,7}]decane. The preparation of (4s)-4-(5-phenyl-1,3,4-thiadiazol-2-yloxy)-1-azatricyclo[3.3.1.1^{3,7}]decane is disclosed in US Patent Application Publication No. 20080167336.

### b. Salts of the nAChR Ligand Modulators

The nAChR ligand modulators described above may exist as therapeutically suitable salts. The term “therapeutically suitable salt,” refers to salts or zwitterions of the compounds, which are water or oil-soluble or dispersible, suitable for treatment of disorders without undue toxicity, irritation, and allergic response, commensurate with a reasonable benefit/risk ratio, and effective for their intended use. The salts may be prepared during the final isolation and purification of the compounds or separately by reacting an amino group of the compounds with a suitable acid. For example, a compound may be dissolved in a suitable solvent, such as but not limited to methanol and water, and treated with at least one equivalent of an acid, like hydrochloric acid. The resulting salt may precipitate out and be isolated by filtration and dried under reduced pressure. Alternatively, the solvent and excess acid may be removed under reduced pressure to provide the salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, form ate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. The amino groups of the compounds may also be quaternized with alkyl chlorides, bromides, and iodides such as methyl, ethyl, propyl, isopropyl, butyl, lauryl, myristyl, stearyl, and the like.

Substantially pure crystalline salts of (4s)-4-(5-phenyl-1,3,4-thiadiazol-2-yloxy)-1-azatricyclo[3.3.1.1^{3,7}]decane are, for example, (4s)-4-(5-phenyl-1,3,4-thiadiazol-2-yloxy)-1-azatricyclo[3.3.1.1^{3,7}]decane L-bitartrate anhydrate, (4s)-4-(5-phenyl-1,3,4-thiadiazol-2-yloxy)-1-azatricyclo[3.3.1.1^{3,7}]decane L-bitartrate hydrate, (4s)-4-(5-phenyl-1,3,4-thiadiazol-2-yloxy)-1-azatricyclo[3.3.1.1^{3,7}]decane dihydrogen phosphate anhydrate, (4s)-4-(5-phenyl-1,3,4-thiadiazol-2-yloxy)-1-azatricyclo[3.3.1.1^{3,7}]decane dihydrogen phosphate hydrate, (4s)-4-(5-phenyl-1,3,4-thiadiazol-2-yloxy)-1-azatricyclo[3.3.1.1^{3,7}]decane bisuccinate anhydrate, (4s)-4-(5-phenyl-1,3,4-thiadiazol-2-yloxy)-1-azatricyclo[3.3.1.1^{3,7}]decane bisuccinate hydrate, (4s)-4-(5-phenyl-1,3,4-thiadiazol-2-yloxy)-1-azatricyclo[3.3.1.1^{3,7}]decane hydrochloride quarterhydrate, (4s)-4-(5-phenyl-1,3,4-thiadiazol-2-yloxy)-1-azatricyclo[3.3.1.1^{3,7}]decane hydrochloride sesquihydrate, (4s)-4-(5-phenyl-1,3,4-thiadiazol-2-yloxy)-1-azatricyclo[3.3.1.1^{3,7}]decane dihydrogen citrate, (4s)-4-(5-phenyl-1,3,4-thiadiazol-2-yloxy)-1-azatricyclo[3.3.1.1^{3,7}]decane monohydrogen citrate, or (4s)-4-(5-phenyl-1,3,4-thiadiazol-2-yloxy)-1-azatricyclo[3.3.1.1^{3,7}]decane.

One particular salt suitable for the invention is (E)-4-[(5-phenyl-1,3,4-thiadiazol-2-yl)oxy]-1-azoniatricyclo[3.3.1.1^{3,7}]decane 3,4-dicarboxy-3-hydroxybutanoate hydrate.

Basic addition salts may be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

### c. Amides, Esters and Prodrugs

The method may use amides, esters or prodrugs of the nAChR receptor ligand modulators. Prodrugs are derivatives of an active drug designed to ameliorate some identified, undesirable physical or biological property. The physical properties are usually solubility (too much or not enough lipid or aqueous solubility) or stability related, while problematic biological properties include too rapid metabolism or poor bioavailability which itself may be related to a physicochemical property.

Prodrugs are usually prepared by: a) formation of ester, hemi esters, carbonate esters, nitrate esters, amides, hydroxamic acids, carbamates, imines, Mannich bases, and enamines of the active drug, b) functionalizing the drug with azo, glycoside, peptide, and ether functional groups, c) use of polymers, salts, complexes, phosphoramides, acetals, hemiacetals, and ketal forms of the drug. For example, see Andrejus Korolkovas's, “Essentials of Medicinal Chemistry”, John Wiley-Interscience Publications, John Wiley and Sons, New York (1988), pp. 97-118, which is incorporated in its entirety by reference herein.

Esters can be prepared from substrates of formula (I) containing either a hydroxyl group or a carboxy group by general methods known to persons skilled in the art. The typical reactions of these compounds are substitutions replacing one of the heteroatoms by another atom, for example:

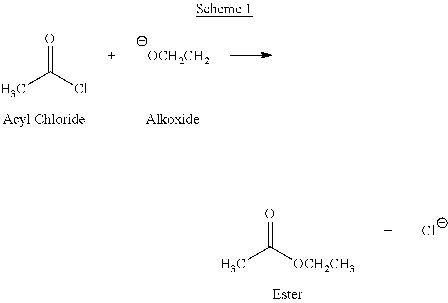

Amides can be prepared from substrates of formula (I) containing either an amino group or a carboxy group in similar fashion. Esters can also react with amines or ammonia to form amides.

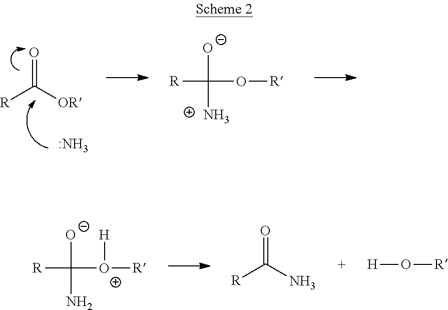

Another way to make amides from compounds of formula (I) is to heat carboxylic acids and amines together.

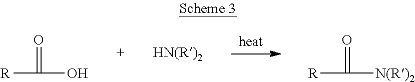

In Schemes 2 and 3, R and R′ are independently substrates of formulas I-V, alkyl or hydrogen.

### d. Optical Isomers, Diastereomers-Geometric Isomers

Asymmetric centers may exist in the nAChR ligand modulators. Individual stereoisomers of the compounds are prepared by synthesis from chiral starting materials or by preparation of racemic mixtures and separation by conversion to a mixture of diastereomers followed by separation or recrystallization, chromatographic techniques, or direct separation of the enantiomers on chiral chromatographic columns. Starting materials of particular stereochemistry are either commercially available or are made by the methods described hereinbelow and resolved by techniques well known in the art.

Geometric isomers may exist in the nAChR ligand modulators. The invention contemplates the various geometric isomers and mixtures thereof resulting from the disposal of substituents around a carbon-carbon double bond, a cycloalkyl group, or a heterocycloalkyl group. Substituents around a carbon-carbon double bond are designated as being of Z or E configuration and substituents around a cycloalkyl or heterocycloalkyl are designated as being of cis or trans configuration. Furthermore, the invention contemplates the various isomers and mixtures thereof resulting from the disposal of substituents around an adamantane ring system. Two substituents around a single ring within an adamantane ring system are designated as being of Z or E relative configuration. For examples, see C. D. Jones, M. Kaselj, R. N. Salvatore, W. J. le Noble J. Org. Chem. 63: 2758-2760, 1998.

The nAChR ligand modulators of the invention may exist as stereoisomers wherein, asymmetric or chiral centers are present. These stereoisomers are “R” or “S” depending on the configuration of substituents around the chiral element. The terms “R” and “S” used herein are configurations as defined in IUPAC 1974 Recommendations for Section E, Fundamental Stereochemistry, Pure Appl. Chem., 1976, 45: 13-30. The invention contemplates various stereoisomers and mixtures thereof and are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

The nAChR ligand modulators may exist in the forms represented by formula (Ia) and (Ib).

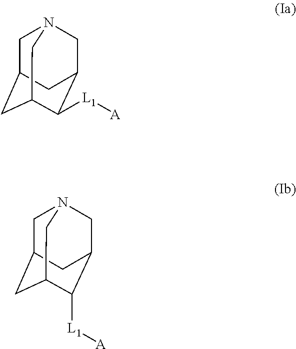

The aza-adamantane portion of isomer (Ia) and isomer (Ib) is not chiral, however the C-4 carbon at which L₁ is attached is considered pseudoasymmetric. Compounds represented by formula (Ia) and (Ib) are diastereomers. The configurational assignment of structures of formula (Ia) are assigned 4r in accordance with that described in Synthesis, 1992, 1080, Becker, D. P.; Flynn, D. L. and as defined in Stereochemistry of Organic Compounds, E. L. Eliel, S. H Wilen; John Wiley and Sons, Inc. 1994. In addition the configurational assignment of structures of formula (Ib) are assigned 4s using the same methods.

The isomers (Ia) and (Ib) may be synthesized separately using the individual stereoisomers according to the Schemes or the Experimentals described herein. Alternatively, isomers (Ia) and (Ib) may be synthesized together after which the individual isomers may be separated by chromatographic methods from the mixture of both isomers when mixtures of stereoisomers are used in the synthesis. The mixtures of isomers may also be separated through fractional crystallization of salts of amines contained in the compounds of formula (I) made with enantiomerically pure carboxylic acids.

It is contemplated that a mixture of both isomers may be used to modulate the effects of nAChRs. Furthermore, it is contemplated that the individual isomers of formula (Ia) and (Ib) may be used alone to modulate the effects of nAChRs. Therefore, it is contemplated that either a mixture of the compounds of formula (Ia) and (Ib) or the individual isomers alone represented by the compounds of formula (Ia) or (Ib) would be effective in modulating the effects of nAChRs, and more particularly α7 nAChRs, α4β2 nAChRs, or a combination of α7 nAChRs and α4β2 nAChRs and is thus within the scope of the invention.

More specifically, the nAChR ligand modulators may include

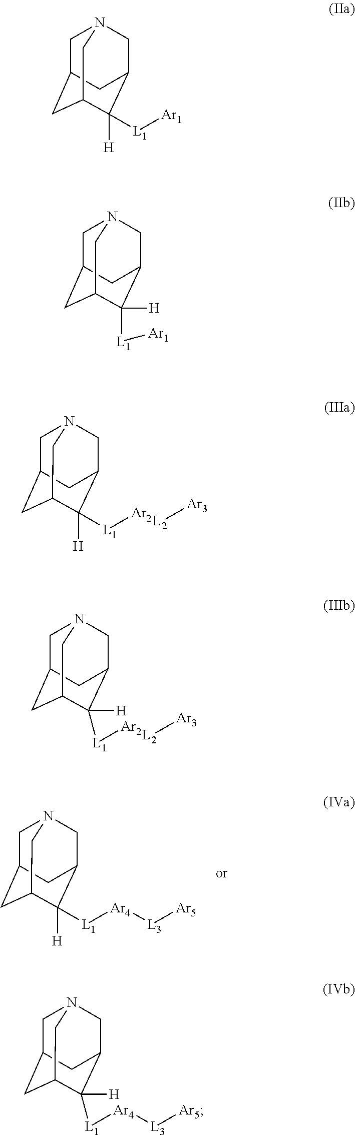

wherein L₁, L₂, L₃, Ar₁, Ar₂, Ar₃, Ar₄, and Ar₅ are defined herein.

### e. Isotope Enriched or Labeled Compounds

The nAChR ligand modulators can exist in isotope-labeled or enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to, ²H, ³H, ¹³C, ¹⁴C, ¹⁵N, ¹⁸O, ³²P, ³⁵S, ¹⁸F, ³⁶Cl and ¹²⁵I. Compounds that contain other isotopes of these and/or other atoms are within the scope of this invention.

In another embodiment, the isotope-labeled compounds contain deuterium (²H), tritium (³H) or ¹⁴C isotopes. Isotope-labeled compounds of this invention can be prepared by the general methods well known to persons having ordinary skill in the art. Such isotope-labeled compounds can be conveniently prepared by carrying out the procedures disclosed in the Examples disclosed herein and Schemes by substituting a readily available isotope-labeled reagent for a non-labeled reagent. In some instances, compounds may be treated with isotope-labeled reagents to exchange a normal atom with its isotope, for example, hydrogen for deuterium can be exchanged by the action of a deuteric acid such as D₂SO₄/D₂O. In addition to the above, relevant procedures and intermediates are disclosed, for instance, in Lizondo, J et al., Drugs Fut, 21(11), 1116 (1996); Brickner, S J et al., J Med Chem, 39(3), 673 (1996); Mallesham, B et al., Org Lett, 5(7), 963 (2003); PCT publications WO1997010223, WO2005099353, WO1995007271, WO2006008754; U.S. Pat. Nos. 7,538,189; 7,534,814; 7,531,685; 7,528,131; 7,521,421; 7,514,068; 7,511,013; and US Patent Application Publication Nos. 20090137457; 20090131485; 20090131363; 20090118238; 20090111840; 20090105338; 20090105307; 20090105147; 20090093422; 20090088416; and 20090082471, the methods are hereby incorporated by reference.

The isotope-labeled nAChR ligand modulators of the invention may be used as standards to determine the effectiveness of nAChR ligands in binding assays. Isotope containing compounds have been used in pharmaceutical research to investigate the in vivo metabolic fate of the compounds by evaluation of the mechanism of action and metabolic pathway of the nonisotope-labeled parent compound (Blake et al. J. Pharm. Sci. 64, 3, 367-391 (1975)). Such metabolic studies are important in the design of safe, effective therapeutic drugs, either because the in vivo active compound administered to the patient or because the metabolites produced from the parent compound prove to be toxic or carcinogenic (Foster et al., Advances in Drug Research Vol. 14, pp. 2-36, Academic press, London, 1985; Kato et al., J. Labelled Comp. Radiopharmaceut., 36(10):927-932 (1995); Kushner et al., Can. J. Physiol. Pharmacol., 77, 79-88 (1999).

In addition, non-radio active isotope containing drugs, such as deuterated drugs called “heavy drugs,” can be used for the treatment of diseases and conditions related to nAChR activity. Increasing the amount of an isotope present in a compound above its natural abundance is called enrichment. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %. Replacement of up to about 15% of normal atom with a heavy isotope has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. New York Acad. Sci. 1960 84: 736; Czakja D M et al., Am. J. Physiol. 1961 201: 357). Acute replacement of as high as 15%-23% in human fluids with deuterium was found not to cause toxicity (Blagojevic N et al. in “Dosimetry & Treatment Planning for Neutron Capture Therapy”, Zamenhof R, Solares G and Harling 0 Eds. 1994. Advanced Medical Publishing, Madison Wis. pp. 125-134; Diabetes Metab. 23: 251 (1997)).

Stable isotope labeling of a drug can alter its physico-chemical properties such as pKa and lipid solubility. These effects and alterations can affect the pharmacodynamic response of the drug molecule if the isotopic substitution affects a region involved in a ligand-receptor interaction. While some of the physical properties of a stable isotope-labeled molecule are different from those of the unlabeled one, the chemical and biological properties are the same, with one important exception: because of the increased mass of the heavy isotope, any bond involving the heavy isotope and another atom will be stronger than the same bond between the light isotope and that atom. Accordingly, the incorporation of an isotope at a site of metabolism or enzymatic transformation will slow said reactions potentially altering the pharmacokinetic profile or efficacy relative to the non-isotopic compound.

### 8. Pharmaceutical Compositions

The methods of the invention described above correlate particular SNP profiles of the COMT gene with particular nAChR ligand modulators. The nAChR ligand modulators may be administered in effective amount in a pharmaceutical composition.

The pharmaceutical composition may comprise an effective amount of an nAChR ligand modulators as described above, or pharmaceutically acceptable salts, prodrugs, esters, amides or metabolites thereof formulated with one or more therapeutically suitable excipients.

a. Effective Amount of Pharmaceutical Composition and nAChR Ligand Modulator

The method may including the step of administering an effective dosage of the particular nAChR ligand modulator to the patient identified as being a candidate for effective treatment for improving cognition impairment. The therapeutically effective amount may comprise an amount of the nAChR ligand modulator from about 6 mg to about 150 mg. The therapeutically effective amount of the nAChR ligand modulator may be selected from the group consisting of about 10 mg to about 150 mg, 10 mg to about 75 mg, about 10 mg to about 50 mg, about 10 mg to about 25 mg, about 25 mg to about 150 mg, about 25 mg to about 75 mg, about 25 mg to about 50 mg, about 25 mg to about 50 mg, or about 50 mg to about 75 mg.

The therapeutically effective amount of composition comprising (4s)-4-(5-phenyl-1,3,4-thiadiazol-2-yloxy)-1-azatricyclo[3.3.1.1^{3,7}]decane (Compound A) may be about 10 mg to about 150 mg. The therapeutically effective amount of Compound A may be selected from the group consisting of about 10 mg to about 150 mg, 10 mg to about 75 mg, about 10 mg to about 50 mg, about 10 mg to about 25 mg, about 25 mg to about 150 mg, about 25 mg to about 75 mg, about 25 mg to about 50 mg, about 25 mg to about 50 mg, or about 50 mg to about 75 mg.

In another embodiment, the therapeutically effective amount of Compound A comprises an amount of the nAChR ligand from about 25 mg to about 75 mg. Compound A may be administered in doses of 10 mg, 25 mg, 50 mg, or 75 mg.

b. Pharmaceutically Acceptable Carrier

The pharmaceutical composition may further comprise a pharmaceutically acceptable carrier. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

c. Administration of the Pharmaceutical Composition/Pharmaceutical Formulations

The method may include administering the pharmaceutical composition to the patient described above. The pharmaceutical composition can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions can also contain adjuvants such as preservative agents, wetting agents, emulsifying agents, and dispersing agents. Prevention of the action of microorganisms can be ensured by various antibacterial and antifungal agents, for example, parabens, chlorobutanol, phenol, sorbic acid, and the like. It also can be desirable to include isotonic agents, for example, sugars, sodium chloride and the like. Prolonged absorption of the injectable pharmaceutical form can be brought about by the use of agents delaying absorption, for example, aluminum monostearate and gelatin.

In some cases, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug can depend upon its rate of dissolution, which, in turn, may depend upon crystal size and crystalline form. Alternatively, a parenterally administered drug form can be administered by dissolving or suspending the drug in an oil vehicle.

Suspensions, in addition to the active compounds, can contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions can be formulated according to the known art using suitable dispersing or wetting agents and suspending agents. The sterile injectable preparation also can be a sterile injectable solution, suspension or emulsion in a nontoxic, parenterally acceptable diluent or solvent such as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Compositions for rectal or vaginal administration are preferably suppositories which can be prepared by mixing the compounds of this invention with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents. Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention.

The ointments, pastes, creams and gels may contain, in addition to an active compound of this invention, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, lactose, talc, silicic acid, aluminum hydroxide, calcium silicates and polyamide powder, or mixtures of these substances. Sprays can additionally contain customary propellants such as chlorofluorohydrocarbons.

Compounds of the invention also can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the invention, stabilizers, preservatives, and the like. The preferred lipids are the natural and synthetic phospholipids and phosphatidylcholines (lecithins) used separately or together. Methods to form liposomes are known in the art. See, for example, Prescott, Ed., Methods in Cell Biology, Volume XIV, Academic Press, New York, N.Y., (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this invention. Aqueous liquid compositions of the invention also are particularly useful.

The nAChR ligand modulators of the invention can be used in the form of pharmaceutically acceptable salts. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Representative acid addition salts can be prepared using various suitable acids for example, including, but are not limited to, acetic, adipic, alginic, citric, aspartic, benzoic, benzenesulfonic, butyric, camphoric, camphorsulfonic, carbonic, digluconic, glycerophosphoric, heptanoic, hexanoic, fumaric, hydrochloric, hydrobromic, hydroiodic, 2-hydroxyethansulfonic (isethionic), lactic, maleic, methanesulfonic, nicotinic, 2-naphthalenesulfonic, oxalic, pamoic, pectinic, persulfuric, 3-phenylpropionic, picric, pivalic, propionic, succinic, sulfuric, tartaric, thiocyanic, phosphoric, glutamatic, p-toluenesulfonic, and undecanoic acids.

Particular examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, tartaric acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The nAChR ligand modulator can be administered in the form of a pharmaceutical composition or compositions that contain one or both active agents in an admixture with a pharmaceutical carrier. The pharmaceutical composition can be in dosage unit form such as tablet, capsule, sprinkle capsule, granule, powder, syrup, suppository, injection, or the like.

All patents, patent applications, and literature references cited in the specification are herein incorporated by reference in their entirety.

For a variable that occurs more than one time in any substituent or in the compound of the invention or any other formulae herein, its definition on each occurrence is independent of its definition at every other occurrence. Combinations of substituents are permissible only if such combinations result in stable compounds. Stable compounds are compounds which can be isolated in a useful degree of purity from a reaction mixture.

### 9. Animal Model of COMT Activity Mouse Strain Differences

The methods of the invention described above correlate particular SNP profiles of the COMT gene with particular nAChR ligand modulators. The nAChR ligand modulators may be administered in effective amount in a pharmaceutical composition.

Polymorphisms in the catechol-O-methyltransferase (COMT) gene may be important for determining treatment regimens in schizophrenia. Therefore, an animal model that utilizes strain differences in COMT activity may be useful for the preclinical determination of treatments for schizophrenia.

It has been shown that mouse strains a length polymorphism of the Comt1 gene in mouse strains such at the C57BL/6J results in higher specific activity of COMT1 in hippocampal protein compared to strains lacking the insertion such as DBA/2J mice. Another strain lacking the B2 insertion is the C57L/J which is very closely related to the C57BL/6J.

The method described herein measures COMT activity in mouse frontal cortex and in washed erythrocytes from C57BL/6J and C57L/J mice. This model has the potential to be utilized to demonstrate responsiveness to clinical candidate compounds. Certain aspects of the invention are described in greater detail in the non-limiting Examples that follow:

### EXAMPLES

### Example 1

### Clinical Study A

### Experimental Details

### Subjects

A Phase 2a proof-of-concept (POC) study in clinically stable subjects with schizophrenia who were clinically stable and received stable doses of atypical antipsychotic therapy. The study was a Phase 2, multi-center, randomized, double-blind, placebo-controlled, parallel group study designed to evaluate the safety and efficacy of doses of (4s)-4-(5-phenyl-1,3,4-thiadiazol-2-yloxy)-1-azatricyclo[3.3.1.1^{3,7}]decane (Compound A) in clinically stable male and female subjects (ages 20 to 55, inclusive) with a Diagnostic and Statistical Manual of Mental Disorders—Fourth Edition, Text Revision (DSM-IV-TR) diagnosis of schizophrenia. Psychiatric diagnoses were confirmed using the Mini-International Neuropsychiatric Interview (MINI) version 6.0.0. The criteria for clinical stability was determined by a combination of retrospective data (over the 4 months prior to the start of Screening, which will be supported by clinical records, patient, identified responsible contact person, and physician interviews), and prospective data assessed during the Prospective Stabilization Period of 28 to 42 days duration.

Study Design

The study was a Phase 2, multi-center, randomized, double-blind, placebo-controlled, parallel group study designed to evaluate the safety and efficacy of doses of (4s)-4-(5-phenyl-1,3,4-thiadiazol-2-yloxy)-1-azatricyclo[3.3.1.1^{3,7}]decane, which is also recognized as Compound A, in treating cognitive deficits in subjects with schizophrenia who were clinically stable and receiving one or two atypical antipsychotic medications. Study drug was administered orally.

The study consisted of a screening period of at least 28 and up to 42 days, a 84-day outpatient treatment period, a 14-day post-treatment period, and a post-treatment follow-up period. The screening period consisted of three visits: Screening Visit 1, Screening Visit 2, and Day −1. Upon completion of Day −1 procedures, eligible subjects were randomized through an Interactive Voice Response/Interactive Web-Based (IVR/IWB) system. Subjects were randomized in an equal ratio to one of three treatment groups (placebo, 10 mg Compound A, or 25 mg Compound A).

Inclusion Criteria for Study Subjects

Study subjects eligible for participation in the study met the following criteria during the screening period:

Male or female between 20 and 55 years of age, inclusive, at the time of randomization (Day −1).

Have a current DSM-IV-TR diagnosis of schizophrenia confirmed by the M.I.N.I. version 6.0.0.

Receiving an antipsychotic regimen of one or two atypical antipsychotic medications.

Is clinically stable in the residual phase of illness, as defined by the following criteria:

Level of Care: The subject had no psychiatric inpatient hospitalization, no overnight crisis stabilization, no emergency room visit for psychiatric symptoms, and no other overt signs of destabilization from 4 months prior to the Initial Screening Visit.

Stability of Medication Regimen: The subject was receiving antipsychotic therapy with one or two atypical antipsychotic medications for at least 8 weeks prior to Day −1 Visit. In addition, the subject had no symptom-related changes in antipsychotic or antidepressant medications from 8 weeks prior to Day −1 and no changes in dose(s) of those medications for any reason from 4 weeks prior to Day −1.

Severity of Symptoms: Core positive symptoms were no worse than moderate in severity, extrapyramidal symptoms (EPS) were no worse than mild in severity, and depressive symptoms are not consistent with a major depressive episode from the start of Screening through the end of the Prospective Stabilization Period, as defined by the following:

Positive and Negative Syndrome Scale (PANSS) item scores of ≦4 each for delusions (P1), conceptual disorganization (P2), hallucinatory behavior (P3), and excitement (P4);

In the Investigator's judgment, no clinically significant EPS at the Initial Screening Visit, a Day −1 Severity of Abnormal Movements item score of ≦2 on the Abnormal Involuntary Movement Scale (AIMS), and a Day −1 Global Clinical Rating of Akathisia item score of ≦2 on the Barnes Akathisia Rating Scale (BAS);

Calgary Depression Scale for Schizophrenia (CDSS) total score of ≦10 at Screening.

Had been diagnosed with or treated for schizophrenia for at least 2 years prior to Screening.

If female, must be either not of childbearing potential, defined as postmenopausal for at least 2 years or surgically sterile (bilateral tubal ligation, bilateral oophorectomy, or hysterectomy), or of childbearing potential and agree to using a double barrier method (physical barrier, e.g., condom or IUD, and chemical barrier, e.g., birth control pills, jellies or foams) from the time of the Initial Screening Visit through the end of the Follow-up Period. Diaphragm must be used with spermicidal foam or jelly. The combination of diaphragm and spermicidal substance counts as a single barrier.

If a female is of childbearing potential, the result of a serum pregnancy test performed at the initial Screening Visit is negative, and the subject does not plan to become pregnant during the study.

If female, is not breast-feeding.

If male, is surgically sterile (vasectomy), is sexually inactive, or agrees to using a barrier method (condom) of birth control from the time of the Initial Screening Visit through the end of the Follow-up Period.

Has had continuity in psychiatric care (mental health system, clinic or physician), as indicated by available medical records or a corroborating clinician or case worker for at least 6 months prior to Screening.

Randomization, Medication Dosing, and Dispensing

Subjects were randomized in a 1:1:1 ratio with placebo, 10 mg QD Compound A, of 25 mg Compound A. Each subject was instructed to take study drug once-daily in the morning for 12 weeks. Each daily dose was preferably taken with food. The subject and investigator were blinded to the treatment assignment throughout the study. The treatment assignments for the study subjects are shown below in Table 1.

TABLE 1
Treatment Assignments

| Treatment Group | N | Dose |
|---|---|---|
| A | 70 | Placebo QD |
| B | 70 | 10 mg Compound A QD |
| C | 70 | 25 mg Compound A QD |

Visits and Measurements

Subjects completed 2 visits during the screening period. Study site personnel contacted each subject by telephone on Day 21, 35, and 70 of the 84-day treatment period to discuss study drug compliance, antipsychotic medication compliance, concomitant medication use, substance use, and any adverse events.

Endpoints and Measures of Outcome

The primary endpoint was the MCCB composite score, and the primary endpoint analysis was the change on the MCCB composite score from baseline to endpoint versus placebo. Other secondary measures included the MCCB domains, the NSA-16, the CANTAB cognition battery (measured at different time points from the MCCB), and the UPSA-2. The Positive and Negative Syndrome Scale (PANSS) was included to document stability in schizophrenia symptomatology.

MATRICS Consensus Cognitive Battery (MCCB)

The MCCB was developed by a consortium of academic, industry, the Food and Drug Administration (FDA) and National Institute of Mental Health (NIMH) members called Measurement and Treatment Research to Improve Cognition in Schizophrenia (MATRICS). The battery was established in a multiple phase process that involved experts identifying cognitive tests in the literature that had shown deficits in schizophrenia, use of factor analysis to identify key domains of cognitive deficits in schizophrenia, and then empirically identifying the best tests for each domain based on reliability, validity and feasibility for use in clinical trials. The FDA has endorsed the MCCB as an appropriate outcome measure for Phase 3 CDS trials.

The MCCB comprises 10 tests (Trail Making Test Part A, Brief Assessment of Cognition in Schizophrenia Symbol Coding, Hopkins Verbal Learning Test—Revised Immediate Recall Three Trial Learning, Wechsler Memory Scale 3^{rd} Ed. Spatial Span, Letter-Number Span, Neuropsychological Assessment Battery Mazes, Brief Visuospatial Memory Test—Revised, Category Fluency Test Animal Naming, Mayer-Solvay-Caruso Emotional Intelligence Test Managing Emotions, Continuous Performance Test Identical Pairs) of cognitive functioning and assesses seven domains of cognition (speed of processing, verbal learning, working memory, reasoning and problem solving, visual learning, attention/vigilance and social cognition). Repeated administration of the MCCB tests of verbal learning, visual learning and reasoning may result in large content-related practice effects. Therefore, alternate versions of these tests were used in order to minimize practice effects. In order to control for alternate form difficulty, the sequence of the alternate forms were counterbalanced across patients so that at study end, each form was given at each visit a similar number of times. Each site received a schedule for alternate forms from NeuroCog Trials. The MCCB showed good test-retest reliability and discriminated patients with schizophrenia from normal subjects and correlates with functional status. The MCCB took approximately 60 to 90 minutes to administer and was given at the times indicated in on Days 14, 28, 56, 84 and 98.

UCSD Performance-Based Skills Assessment-2 (UPSA-2)

The UCSD Performance-Based Skills Assessment-2 (UPSA-2) is a role-play test designed for subjects with schizophrenia to evaluate cognitive functional capacity in six selected domains of basic living skills. These areas include Organization/Planning, Financial Skills, Communication, Transportation, Household Management, and Medication Management. Patients being tested utilize props to demonstrate how they perform everyday activities and are assessed on their actual performance. Scores were obtained for each subtest, and the total score was the sum of these subtests. The UPSA-2 demonstrated established reliability and validity and significantly correlated with the MCCB. The UPSA-2 required an average of 30 minutes to administer. The UPSA-2 was administered in on Days 14, 28, 56, 84 and 98.

Cambridge Neuropsychological Test Automated Battery (CANTAB) for Schizophrenia

The Cambridge Neuropsychological Test Automated Battery (CANTAB) is a computer-based cognitive assessment system consisting of a battery of neuropsychological tests, administered to subjects using a touch screen computer. The CANTAB battery shows good test/retest reliability and discriminates patients with schizophrenia from normal subjects. The battery also shows pharmacologic sensitivity to a number of compounds including atomoxetine. The CANTAB computerized system will be employed to explore the effects of Compound A on cognition. The tests assess the following cognitive domains: executive function, spatial memory, attention and episodic memory. The CANTAB battery took approximately 40 minutes to administer and was given on Days 14, 28, 56, 84 and 98.

The cognitive tests included in this version of the CANTAB battery are as follows:

Motor Screening

Rapid Visual Information Processing

5 Choice Serial Reaction Time

Spatial Working memory

Paired Associates Learning

Stockings of Cambridge

Emotion Recognition Task

Delayed Match to Sample

Statistical Analysis

Individual Compound A plasma concentrations at each study visit were tabulated and summarized with appropriate statistical methods. Population pharmacokinetic analyses were performed using the actual sampling time relative to dosing. Pharmacokinetic models were built using a non-linear mixed-effect modeling (NONMEM) approach with the NONMEM software (Version VI, or higher version). The structure of the starting pharmacokinetic model will be based on the pharmacokinetic analysis of data from previous studies. Apparent oral clearance (CL/F) and apparent volume of distribution (Vₛₛ/F) of Compound A were the pharmacokinetic parameters of major interest in the NONMEM analyses. If necessary, other parameters, including the parameters describing absorption characteristics, were fixed in the analysis.

Results

Subject Characteristics and Disposition

A total of 207 subjects were randomized. Four subjects did not receive a study drug following randomizations and were not included in the efficacy analyses. The disposition of subjects is shown in Table 2.

TABLE 2
Preliminary Disposition of Subjects

| | Treatment Group, n (%) | | | | |
|---|---|---|---|---|---|
| | | Compound A | | | |
| Subjects: | Placebo | 10 mg QD | 25 mg QD | Overall | Total |
| Random- | 68 | 70 | 69 | 139 | 207 |
| ized | | | | | |
| Treated | 67 | 69 | 67 | 136 | 203 |
| Completed | 56 (83.6) | 60 (87.0) | 49 (73.1) | 109 (80.1) | 165 (81.3) |
| study | | | | | |
| Primary | 11 (16.4) | 9 (13.0) | 18 (26.9) | 27 (19.9) | 38 (18.7) |
| reason for | | | | | |
| discontin- | | | | | |
| uation | | | | | |
| Adverse | 5 (7.5) | 4 (5.8) | 3 (4.5) | 7 (5.1) | 12 (5.9) |
| event | | | | | |
| Withdrew | 2 (3.0) | 1 (1.4) | 3 (4.5) | 4 (2.9) | 6 (3.0) |
| consent | | | | | |
| Lost to | 1 (1.5) | 1 (1.4) | 4 (6.0) | 5 (3.7) | 6 (3.0) |
| follow-up | | | | | |
| Other | 3 (4.5) | 3 (4.3) | 8 (11.9) | 11 (8.1) | 14 (6.9) |

Note:
Percentages are based on the number of treated subjects.

One hundred sixty-five (81.3%) of the treated subjects completed the study. Of the subjects who prematurely terminated, 12 (5.9%) were primarily discontinued due to adverse events and 14 (6.9%) were primarily discontinued for other reasons. The Compound A 25 mg group had the lowest completion rate, with 73.1%. The imbalance was largely in the “other” category; the “other” reasons in this treatment group were heterogeneous, and no clear pattern could be ascribed to the discontinuations in the category. Baseline characteristics of the patients are shown in Table 3.

TABLE 3

| | Overall |
|---|---|
| Characteristic | N = 203 |
| Age (years), mean | 42.3 |
| Gender, male, n (%) | 64.5% |
| Average age onset of symptoms | 22.3 years |
| Average age at diagnosis | 25.7 years |
| MATRICS Cognition Consensus Battery Score | 27.4 |
| (mean) | |
| University of California Performance-based Skills | 86.3 |
| Assessment Score (Mean) | |
| Positive and Negative Syndrome Scale Score (mean) | 64.2 |

### Efficacy and Safety

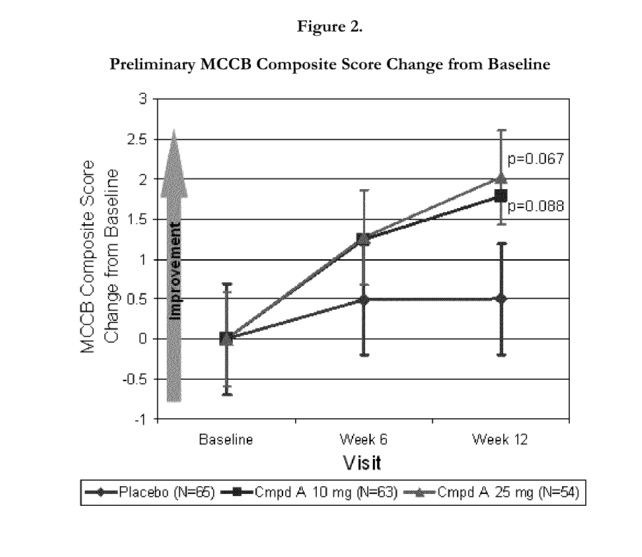

The mean baseline MCCB composite score in this study was 27.4 (SD 12.77) (the scoring has been standardized such that the mean [SD] value in a healthy population is 50 [10]). In the intent-to-treat (ITT) analysis, the change from baseline to Week 12 in MCCB composite score for the Compound A 10 mg and 25 mg dose groups (LS mean+1.79 and +2.02, respectively) trended towards improvement (P=0.088 and P=0.067, respectively) versus placebo (LS mean+0.50) (see FIG. 2)). The results on the composite score were driven by 3 domains: verbal learning (P=0.063 in 25 mg group); working memory (P=0.054 in 25 mg group) and attention (P=0.036 in 25 mg group). In addition, dose response relationships were observed for 6 of the 7 domains; with the lone exception being reasoning. At Week 6, the LS mean difference versus placebo was greater for in both Compound A dose groups for 5 of the 7 MCCB domains (Table 4). The results were consistent on the CANTAB battery.

TABLE 4
Repeated-Measure Analysis of Change from Baseline to Week 6
and Week 12 for MCCB Domain Scores (ITT Data Set)

| | | | LS Mean (SE) | | | | |
|---|---|---|---|---|---|---|---|
| MCCB Domain | | | of Change | Difference from Placebo | | | |
| Visit | | Observed | from | LS Mean (SE) | | | |
| Treatment | N | Mean (SD) | Baseline | of Difference | 90% CI | P value^{a} | |
| Speed of Processing | | | | | | | |
| Baseline | | | | | | | |
| Placebo | 65 | 29.63 (12.05) | | | | | |
| Compound A | 63 | 34.89 (13.86) | | | | | |
| 10 mg | | | | | | | |
| Compound A | 54 | 34.56 (10.97) | | | | | |
| 25 mg | | | | | | | |
| Change to Week 6 | | | | | | | |
| Placebo | 65 | 1.22 (7.02) | 0.40 (0.74) | | | | |
| Compound A | 63 | 1.00 (5.49) | 1.13 (0.74) | 0.74 (1.04) | (−0.98, 2.45) | 0.239 | |
| 10 mg | | | | | | | |
| Compound A | 54 | 2.56 (5.36) | 2.24 (0.80) | 1.84 (1.08) | (0.06, 3.63) | 0.045 | * |
| 25 mg | | | | | | | |
| Change to Week 12 | | | | | | | |
| Placebo | 56 | 1.66 (6.91) | 1.28 (0.87) | | | | |
| Compound A | 60 | 2.33 (6.05) | 2.36 (0.85) | 1.08 (1.20) | (−0.91, 3.07) | 0.185 | |
| 10 mg | | | | | | | |
| Compound A | 49 | 1.73 (7.28) | 1.66 (0.93) | 0.37 (1.26) | (−1.72, 2.47) | 0.384 | |
| 25 mg | | | | | | | |
| Verbal Learning | | | | | | | |
| Baseline | | | | | | | |
| Placebo | 65 | 35.14 (7.63) | | | | | |
| Compound A | 63 | 35.35 (7.57) | | | | | |
| 10 mg | | | | | | | |
| Compound A | 54 | 37.35 (9.55) | | | | | |
| 25 mg | | | | | | | |
| Change to Week 6 | | | | | | | |
| Placebo | 65 | 0.82 (5.91) | 0.07 (0.80) | | | | |
| Compound A | 63 | 0.68 (7.99) | 0.31 (0.82) | 0.24 (1.12) | (−1.62, 2.10) | 0.416 | |
| 10 mg | | | | | | | |
| Compound A | 54 | 0.41 (9.24) | 0.56 (0.88) | 0.49 (1.18) | (−1.47, 2.45) | 0.339 | |
| 25 mg | | | | | | | |
| Change to Week 12 | | | | | | | |
| Placebo | 56 | 1.18 (6.80) | 0.47 (0.80) | | | | |
| Compound A | 60 | 1.28 (7.06) | 1.08 (0.79) | 0.61 (1.10) | (−1.21, 2.43) | 0.290 | |
| 10 mg | | | | | | | |
| Compound A | 49 | 2.24 (8.03) | 2.27 (0.87) | 1.80 (1.17) | (−0.14, 3.47) | 0.063 | + |
| 25 mg | | | | | | | |
| Working Memory | | | | | | | |
| Baseline | | | | | | | |
| Placebo | 65 | 32.52 (11.79) | | | | | |
| Compound A | 63 | 34.43 (11.11) | | | | | |
| 10 mg | | | | | | | |
| Compound A | 54 | 36.09 (10.55) | | | | | |
| 25 mg | | | | | | | |
| Change to Week 6 | | | | | | | |
| Placebo | 65 | −0.23 (5.52) | −0.51 (0.74) | | | | |
| Compound A | 63 | 0.27 (5.15) | 0.35 (0.75) | 0.86 (1.03) | (−0.84, 2.56) | 0.201 | |
| 10 mg | | | | | | | |
| Compound A | 54 | 1.69 (7.43) | 1.88 (0.81) | 2.39 (1.08) | (0.60, 4.18) | 0.014 | + |
| 25 mg | | | | | | | |
| Change to Week 12 | | | | | | | |
| Placebo | 56 | 0.29 (5.76) | 0.11 (0.86) | | | | |
| Compound A | 60 | 0.83 (7.04) | 0.92 (0.84) | 0.80 (1.18) | (−1.15, 2.76) | 0.249 | |
| 10 mg | | | | | | | |
| Compound A | 49 | 1.98 (6.84) | 2.14 (0.93) | 2.03 (1.25) | (−0.04, 4.11) | 0.054 | + |
| Reasoning and | | | | | | | |
| Problem Solving | | | | | | | |
| Baseline | | | | | | | |
| Placebo | 65 | 39.17 (9.43) | | | | | |
| Compound A | 63 | 41.16 (9.78) | | | | | |
| 10 mg | | | | | | | |
| Compound A | 54 | 40.89 (8.87) | | | | | |
| 25 mg | | | | | | | |
| Change to Week 6 | | | | | | | |
| Placebo | 65 | 0.00 (5.56) | −0.04 (0.74) | | | | |
| Compound A | 63 | 0.08 (5.57) | 0.49 (0.75) | 0.53 (1.03) | (−1.18, 2.23) | 0.305 | |
| 10 mg | | | | | | | |
| Compound A | 54 | 2.11 (7.06) | 2.18 (0.81) | 2.22 (1.08) | (0.44, 4.01) | 0.021 | * |
| 25 mg | | | | | | | |
| Change to Week 12 | | | | | | | |
| Placebo | 56 | 0.61 (7.78) | 0.75 (0.85) | | | | |
| Compound A | 60 | 1.70 (6.03) | 1.99 (0.84) | 1.24 (1.18) | (−0.71, 3.19) | 0.147 | |
| 10 mg | | | | | | | |
| Compound A | 49 | 2.00 (6.25) | 1.95 (0.92) | 1.20 (1.24) | (−0.86, 3.25) | 0.169 | |
| Visual Learning | | | | | | | |
| Baseline | | | | | | | |
| Placebo | 65 | 34.32 (12.65) | | | | | |
| Compound A | 63 | 38.30 (12.00) | | | | | |
| 10 mg | | | | | | | |
| Compound A | 54 | 39.37 (12.90) | | | | | |
| 25 mg | | | | | | | |
| Change to Week 6 | | | | | | | |
| Placebo | 65 | 0.75 (9.41) | −0.15 (1.12) | | | | |
| Compound A | 63 | 1.35 (9.36) | 1.59 (1.13) | 1.74 (1.57) | (−0.85, 4.34) | 0.134 | |
| 10 mg | | | | | | | |
| Compound A | 54 | −1.06 (9.04) | −1.15 (1.22) | −1.00 (1.65) | (−3.73, 1.72) | 0.729 | |
| 25 mg | | | | | | | |
| Change to Week 12 | | | | | | | |
| Placebo | 56 | 0.46 (10.89) | −0.44 (1.15) | | | | |
| Compound A | 60 | 0.05 (8.06) | 0.29 (1.12) | 0.73 (1.59) | (−1.90, 3.36) | 0.324 | |
| 10 mg | | | | | | | |
| Compound A | 49 | 1.24 (8.49) | 0.83 (1.23) | 1.27 (1.68) | (−1.51, 4.05) | 0.226 | |
| Attention/Vigilance | | | | | | | |
| Baseline | | | | | | | |
| Placebo | 65 | 35.28 (11.67) | | | | | |
| Compound A | 63 | 36.30 (13.35) | | | | | |
| 10 mg | | | | | | | |
| Compound A | 54 | 37.02 (11.80) | | | | | |
| 25 mg | | | | | | | |
| Change to Week 6 | | | | | | | |
| Placebo | 65 | −0.25 (7.26) | −0.80 (0.96) | | | | |
| Compound A | 63 | 0.83 (7.26) | 0.49 (0.98) | 1.29 (1.35) | (−0.93, 3.52) | 0.169 | |
| 10 mg | | | | | | | |
| Compound A | 54 | 1.09 (9.04) | 0.91 (1.06) | 1.71 (1.41) | (−0.63, 4.05) | 0.114 | |
| 25 mg | | | | | | | |
| Change to Week 12 | | | | | | | |
| Placebo | 56 | −1.52 (7.52) | −1.99 (1.08) | | | | |
| Compound A | 60 | 1.15 (8.46) | 0.64 (1.06) | 2.63 (1.49) | (0.17, 5.09) | 0.040 | * |
| 10 mg | | | | | | | |
| Compound A | 49 | 0.88 (9.09) | 0.86 (1.16) | 2.85 (1.57) | (0.25, 5.45) | 0.036 | * |
| Social Cognition | | | | | | | |
| Baseline | | | | | | | |
| Placebo | 65 | 35.98 (13.48) | | | | | |
| Compound A | 63 | 35.83 (12.82) | | | | | |
| 10 mg | | | | | | | |
| Compound A | 54 | 38.02 (13.81) | | | | | |
| 25 mg | | | | | | | |
| Change to Week 6 | | | | | | | |
| Placebo | 65 | 1.48 (6.95) | 1.50 (0.95) | | | | |
| Compound A | 63 | 1.14 (7.26) | 1.04 (0.97) | −0.46 (1.34) | (−2.67, 1.75) | 0.636 | |
| 10 mg | | | | | | | |
| Compound A | 54 | 0.06 (8.00) | 0.30 (1.05) | −1.21 (1.40) | (−3.53, 1.11) | 0.805 | |
| 25 mg | | | | | | | |
| Change to Week 12 | | | | | | | |
| Placebo | 56 | 0.43 (7.87) | 0.59 (1.10) | | | | |
| Compound A | 60 | 1.38 (6.51) | 0.99 (1.08) | 0.40 (1.52) | (−2.11, 2.92) | 0.395 | |
| 10 mg | | | | | | | |
| Compound A | 49 | −0.14 (10.81) | 0.19 (1.19) | −0.40 (1.61) | (−3.06, 2.27) | 0.597 | |

MCCB = MATRICS Consensus Cognitive Battery,
SD = standard deviation,
LS = least squares,
SE = standard error,
CI = confidence interval
^{a}One-sided P value from repeated measures model with treatment, site, visit, baseline score, interactions of treatment and visit, and interaction of baseline score and visit; covariance structure is unstructured.
Note:
The MCCB domain scores are age- and gender-adjusted T-scores with a population mean of 50 and standard deviation of 10. An increasing MCCB domain score represents improvement from baseline.

Early improvement in cognition with Compound A is suggested by an increase from baseline to Week 6 in MCCB composite score for both the 10 mg group (LS mean+1.25) and the mg dose group (LS mean+1.27) relative to placebo (LS mean+0.49) according to MMRM analysis, although the difference between each Compound A dose group and placebo did not reach the level of statistical significance

No significance was found for either dose group on the UPSA-2 composite score; however, a statistically significant improvement or statistical trend for improvement was seen on the medication management (P=0.094 at 10 mg), comprehension/planning (P=0.102 at 10 mg and P=0.005 at 25 mg), and household skills (P=0.096 at 10 mg) subscale scores (Table 5).

TABLE 5
Analysis of Covariance of Change from Baseline to Final Evaluation for UPSA-2
Total Score

| | | | | | Difference from Placebo | | |
|---|---|---|---|---|---|---|---|
| | | | | LS Mean | LS Mean | | |
| Treatment | | Observed Mean (SD) | | (SE) | (SE) | | |
| Group | N | Baseline | Final | of Change | of Difference | 90% CI | Pvalue^{a} |
| Placebo | 60 | 80.18 (18.70) | 82.60 (19.57) | 2.27 (1.57) | | | |
| Compound A | 62 | 85.94 (13.68) | 87.98 (20.36) | 2.78 (1.52) | 0.51 (2.15) | (−3.04, 4.07) | 0.406 |
| 10 mg | | | | | | | |
| Compound A | 52 | 92.17 (13.16) | 94.29 (15.85) | 2.89 (1.70) | 0.63 (2.34) | (−3.24, 4.49) | 0.394 |
| 25 mg | | | | | | | |

### Example 2

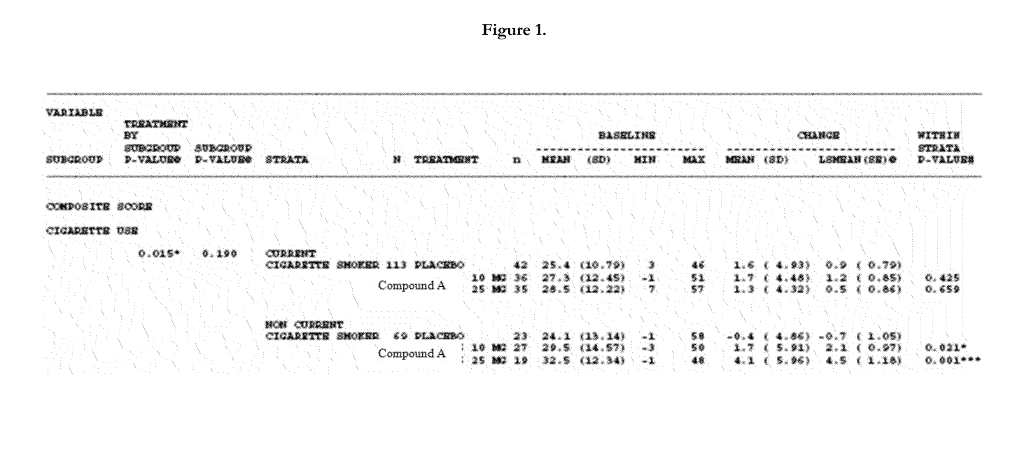

Analysis of the data by cigarette smoking status (current smoker or current nonsmoker) indicated a significant drug-by-treatment interaction (p=0.015). The data indicate a robust treatment effect with a dose-response in the population of subjects who were not current smokers (FIG. 1), with a change score of +2.1 and +4.5 on the MCCB composite score for the Compound A 10 mg and Compound A 25 mg dose groups, respectively (p=0.021 and 0.001, respectively), whereas the change from baseline in the population of smokers was similar for both active Compound A treatment groups.

Further analysis of the MCCB results in the subset of subjects who were current smokers indicated an efficacy signal in subjects who possess the minor allele on any one of four COMT SNPs (Table 6). A blood sample for genotyping was collected from each subject who consented. Genomic DNA was isolated from whole blood using the FlexiGene DNA AGF3000 kit (Qiagen, Valencia, Calif.), on an AutogenFlex 3000 (AutoGen, Holliston, Mass.). Genotypes were determined using the Pyrosequencing detection method (USA—Qiagen, Inc., Valencia, Calif.). Individuals performing the genotyping were blinded to clinical trial data. The significance of the minor allele against the major allele is shown in column marked “Treatment genotype p-value” (Tables 6 and 7). This shows if there is a significant difference in response to Compound A (“Comp. A”) in individuals with the minor allele or heterozygoes versus those with the major allele. For example, for SNP rs4818, individuals with a GC or GG allele have a statistically significantly different response to Compound A relative to individuals with a CC allele.

The SNPs analyzed were RS6269, RS4633, RS4680, and RS4818. The treatment effects across all 4 SNPs in the minor allele indicated a dose-response relationship. The magnitude of the effect for the Compound A 25 mg treatment group was as low as +1.8 points on the MCCB composite score for SNP4680 (corresponding to a Coen's d effect size of 0.43) to as high as +3.7 points for SNP RS4818 (corresponding to a Coen's d effect size of 0.93). No treatment effect was observed in the same population who contained the major allele for these 4 SNPs. In fact, the treatment response for both Compound A active treatment groups was generally less than that of placebo for subjects containing the major COMT allele.

A similar dose-response trend was observed across the four COMT SNPs in the population of nonsmokers as well (data not shown). In this population, while the treatment effect was highly robust for the minor allele, there was also a meaningful effect in subjects containing the major allele as well.

Inspection of the individual responses indicate he results of the genotype analysis did not appear to be driven by outliers.

TABLE 6
Analysis of MCCB in Current Smokers by COMT Allele Type

| | | | | | Change from | Between- | | | Compound |
|---|---|---|---|---|---|---|---|---|---|
| | | | | Baseline | Baseline to | group | | Trt* | A vs PBO |
| | | Treatment | | Mean | Final Mean | Difference | Effect Size | genotype | contrast p- |
| SNP | Genotype | Group | N | (SD) | (SD) | (Pooled SD) | (Raw mean) | p-value | value# |
| RS6269 | G/G or | Placebo | 19 | 24.3(11.3) | 1.1(5.6) | | | 0.203 | |
| Minor | A/G | Compound A | 18 | 26.6(13.0) | 2.9(4.2) | 1.8(5.0) | 0.36 | | |
| allele = | | 10 mg | | | | | | | |
| G | | Compound A | 14 | 28.8(12.9) | 3.1(4.4) | 2.0(5.1) | 0.40 | | |
| | | 25 mg | | | | | | | |
| | A/A | Placebo | 12 | 23.2(11.2) | 1.8(3.0) | −1.0(3.9) | — | | |
| | | Compound A | 13 | 28.7(11.9) | 0.8(4.6) | | | | |
| | | 25 mg | | | | | | | |
| RS4633 | T/T or | Placebo | 19 | 22.9 (11.8) | 0.9(4.1) | | | 0.012 | |
| Minor | C/T | Compound A | 19 | 28.9(13.1) | −0.3(4.4) | −1.2(4.2) | — | | 0.971 |
| allele = | | 10 mg | | | | | | | |
| T | | Compound A | 17 | 28.5 (12.7) | 2.9(4.8) | 2.0(4.5) | 0.44 | | 0.183 |
| | | 25 mg | | | | | | | |
| | C/C | Placebo | 13 | 25.9(9.8) | 1.5(5.7) | −1.1(5.0) | — | | 0.620 |
| | | Compound A | 10 | 29.2(12.0) | 0.4(3.9) | | | | |
| | | 25 mg | | | | | | | |
| RS4680 | A/A or | Placebo | 16 | 23.8(11.6) | 1.1(3.3) | | | 0.044 | |
| Minor | G/A | Compound A | 19 | 28.7(13.3) | 0.1(4.6) | −1.0(4.1) | — | | 0.913 |
| allele = | | 10 mg | | | | | | | |
| A | | Compound A | 17 | 28.5(12.7) | 2.9(4.8) | 1.8(4.2) | 0.43 | | 0.143 |
| | | 25 mg | | | | | | | |
| | G/G | Placebo | 16 | 24.4(10.7) | 1.3(6.0) | −0.9(5.3) | — | | 0.723 |
| | | Compound A | 10 | 29.2(12.0) | 0.4(3.9) | | | | |
| | | 25 mg | | | | | | | |
| RS4818 | G/C or | Placebo | 13 | 25.2(11.6) | −0.3(3.6) | | | 0.081 | |
| Minor | G/G | Compound A | 13 | 28.0(12.6) | 1.7(3.7) | 2.1(3.6) | 0.58 | | 0.135 |
| allele = | | 10 mg | | | | | | | |
| G | | Compound A | 10 | 31.0(14.5) | 3.4(4.4) | 3.7(4.0) | 0.93 | | 0.018 |
| | | 25 mg | | | | | | | |
| | C/C | Placebo | 19 | 23.4(10.8) | 2.2(5.2) | −1.0(4.9) | — | | 0.724 |
| | | Compound A | 17 | 27.4(10.9) | 1.2(4.6) | | | | |
| | | 25 mg | | | | | | | |

TABLE 7
MCCB: Non-smokers
Effect-sizes for different SNPs in COMT gene (RS4680, RS6269, RS4818, RS4633)

| | | | | | Change | | | |
|---|---|---|---|---|---|---|---|---|
| | | | | | from | Between- | Effect | |
| | | | | | Baseline to | group | Size | |
| | | Treatment | | Baseline | Final | Difference | (Raw | Trt* genotype |
| SNP | Genotype | Group | N | Mean(SD) | Mean(SD) | (Pooled SD) | mean) | p-value |
| RS6269 | G/G or A/G | Placebo | 7 | 31.3(13.4) | −0.9(4.6) | 1.9(3.9) | 0.49 | 0.829 |
| Minor | | Compound A | 11 | 33.3(14.4) | 1.0(3.5) | | | |
| allele = | | 10 mg | | | | | | |
| G | | Compound A | 11 | 31.3(15.4) | 5.9(5.6) | 6.8(5.2) | 1.31 | |
| | | 25 mg | | | | | | |
| | A/A | Placebo | 7 | 16.7(11.7) | −0.3(3.0) | 2.2(4.9) | 0.45 | |
| | | Compound A | 7 | 33.9(7.4) | 1.9(6.3) | | | |
| | | 25 mg | | | | | | |
| RS4633 | T/T or C/T | Placebo | 8 | 23.3(15.3) | −0.3(4.0) | 2.7(6.1) | 0.44 | 0.274 |
| Minor | | Compound A | 16 | 30.7(15.9) | 2.4(7.3) | | | |
| allele = | | 10 mg | | | | | | |
| T | | Compound A | 11 | 36.5(9.1) | 2.4(5.4) | 2.7(4.9) | 0.55 | |
| | | 25 mg | | | | | | |
| | C/C | Placebo | 6 | 25.3(14.1) | −1.0(3.6) | 8.4(5.1) | 1.65 | |
| | | Compound A | 7 | 25.6(15.2) | 7.4(6.1) | | | |
| | | 25 mg | | | | | | |
| RS4680 | A/A or G/A | Placebo | 9 | 22.0(14.6) | −0.2(3.7) | 2.4(6.4) | 0.38 | 0.289 |
| Minor | | Compound A | 15 | 29.5(15.7) | 2.2(7.5) | | | |
| allele = | | 10 mg | | | | | | |
| A | | Compound A | 11 | 36.5(9.1) | 2.4(5.4) | 2.6(4.7) | 0.55 | |
| | | 25 mg | | | | | | |
| | G/G | Placebo | 6 | 25.3(14.1) | −1.0(3.6) | 8.4(5.1) | 1.65 | |
| | | Compound A | 7 | 25.6(15.2) | 7.4(6.1) | | | |
| | | 25 mg | | | | | | |
| RS4818 | G/C or G/G | Placebo | 9 | 29.8(13.1) | −0.6(4.0) | 1.8(3.9) | 0.46 | 0.951 |
| Minor | | Compound A | 10 | 35.2(15.4) | 1.2(3.8) | | | |
| allele = | | 10 mg | | | | | | |
| G | | Compound A | 12 | 31.8(14.8) | 5.8(5.3) | 6.4(4.8) | 1.33 | |
| | | 25 mg | | | | | | |
| | C/C | Placebo | 6 | 13.7(9.4) | −0.5(3.5) | 1.8(5.3) | 0.34 | |
| | | Compound A | 6 | 33.2(7.9) | 1.3(6.7) | | | |
| | | 25 mg | | | | | | |

#: one-sided within strata p-values from ANCOVA are only presented if Trt*genotype p-vaue <= 0.1.

### Example 3

### Experimental Details

Clinical Study B: A Randomized, Double-Blind, Placebo-Controlled Dose-Ranging, Parallel-Group, Phase 2 Study of the Efficacy and Safety of Compound A in Treatment of Cognitive Deficits of Schizophrenia (CDS)

This is a multicenter, randomized, double-blind, placebo-controlled, dose-ranging, parallel-group, study designed to evaluate the efficacy and safety of Compound A in the treatment of cognitive deficits in schizophrenia (CDS) in nonsmokers. Approximately 350 subjects will be randomized to one of four treatment groups (Compound A 25 mg, Compound A 50 mg, Compound A 75 mg, or placebo) for a 24 week double-blind treatment period.

Inclusion Criteria for the study subjects include:

1. Male or female between 20 and 55 years of age, inclusive, at the time of randomization.

2. Has a current DSM-IV-TR diagnosis of schizophrenia confirmed by the M.I.N.I.

3. Is receiving one or two antipsychotic medications, restricted to any of the following allowable agents: amisulpride, aripiprazole, asenapine, lurasidone, olanzapine, paliperidone, quetiapine, risperidone, ziprasidone, haloperidol, fluphenazine and perphenazine.

4. Is clinically stable in the residual phase of the illness, as defined by the following criteria:
Level of Care: The subject has had no psychiatric inpatient hospitalization, no overnight crisis stabilization, no emergency room visit for psychiatric symptoms, and no other overt signs of destabilization in the 4 months prior to Screening Visit 1.
Stability of Medication Regimen: The subject has had no symptom-related changes in antipsychotic, antidepressant, or mood-stabilizing medications within 8 weeks prior to Day −1 and no changes in dose(s) of those medications for any reason within 4 weeks prior to Day −1.
Severity of Symptoms: Core positive symptoms are no worse than moderate in severity, extrapyramidal symptoms (EPS) are no worse than mild in severity, and depressive symptoms are not consistent with a major depressive episode from the start of Screening through the end of the Prospective Stabilization Period, as defined by the following: Positive and Negative Syndrome Scale (PANSS) item scores of ≦5 each for delusions (P1), conceptual disorganization (P2), hallucinatory behavior (P3), and excitement (P4); In the Investigator's judgment, no clinically significant EPS at Screening Visit 1, a Severity of Abnormal Movements item score of ≦2 on the Abnormal Involuntary Movement Scale (AIMS) at Day −1, and a Global Clinical Rating of Akathisia score of ≦2 on the Barnes Akathisia Rating Scale (BAS) at Day −1; Calgary Depression Scale for Schizophrenia (CDSS) total score of ≦10 at Screening Visit 1.

5. Has been diagnosed with or treated for schizophrenia for at least 2 years prior to Screening Visit 1.

Exclusion Criteria for the study subjects include:

1. In the Investigator's judgment, has a current or past diagnosis of schizoaffective disorder, bipolar disorder, manic episode, dementia, post traumatic stress disorder, or obsessive-compulsive disorder, or the subject has a current major depressive episode.

2. Has a positive urine drug screen for cocaine, phencyclidine (PCP), opiates (unless duly prescribed), benzodiazepines (unless duly prescribed), marijuana, or amphetamines at Screening Visit 1, Screening Visit 2 or Day −1.

3. Has a body mass index (BMI)>40 kg/m2 at Screening Visit 1. BMI is calculated as weight in kilograms divided by the square of height in meters (kg/m2).

4. Has a current or past history of seizures, with the exception of a single febrile seizure occurring prior to 6 years of age.

5. Has a clinically significant abnormal ECG at Screening Visit 1 as determined by the Investigator.

6. Has any risk factors for Torsades de Pointes (TdP)

Based on the data available for Compound A, it is anticipated that doses of 50 mg QD and 75 mg QD will demonstrate efficacy in the tested subjects as effectively or more effectively than a 25 mg QD dose of Compound A.

In summary, Compound A has demonstrated a signal for efficacy in the symptomatic treatment of AD in the Phase 2a study and appears to be well tolerated in subjects with schizophrenia in doses up to 25 mg QD, including 10 mg QD and 25 mg QD, and can be anticipated to demonstrate efficacy in improving cognitive deficits of schizophrenia at doses of 50 mg QD and 75 mg QD.

### Example 4

### Animal Model of COMT Activity Mouse Strain Differences

The method utilizes [³H]-S-adenosylmethionine as a substrate for the transmethyation of carbachol to methylcarbachol by COMT. The labeled methylcarbachol is then extracted with an organic scintillation cocktail. The method is based on Chen, et al., Am. J. Hum. Genet. 75:807-821, 2004. The method was modified by adding AdoHcy Nucleosidase (EC 3.2.2.9) to remove feedback inhibitor (Hendricks et al. Anal. Biochem. 326:100-105, 2004).

Mice, C57BL/6J or C57L/J, were anesthetized with gaseous CO₂ and blood was drawn either by tail vein or heart puncture and collected in EDTA-treated tubes on ice. Following brain dissection, blood samples were centrifuged at 500×g for 15 min, the plasma was removed, and the erythrocytes were washed twice with Na-phosphate buffer, pH 7.4, and stored at −80° C. The brains were rapidly removed and approximately 25 mg of the frontal cortex was dissected, placed in tubes, frozen on dry ice, and stored at −80° C.

On the day of the assay, the frontal cortex samples were homogenized in homogenization buffer (25 mM Tris-Cl, pH 7.4, 50% glycerol and protease inhibitor cocktail) to yield 50 mg/ml w/v. 20 μl aliquots per assay tube were used. Erythrocytes were thawed and 50 μl samples were lysed 1:10 with 0.1 mg/ml dithiothreitol. 50 μl aliquots per assay tube were used.

Duplicate or triplicate samples of tissue homogenates or erythrocyte lysates, along with samples with 5 μl of 10 mg/ml tropolone to measure nonspecific activity, were preincubated for 10 minutes at 37° C. Incubation for 20 minutes at 37° C. was initiated with the addition of 500 μl of pre-warmed substrate. The substrate consisted of 10 μM pyrocatechol, 200 nM [³H]—S-adenosylmethionine, and 20 nM AdoHcy Nucleosidase (EC 3.2.2.9) in a buffer of 10 mM Tris-Cl, 1 mM MgCl₂, and 10 μM dithiothreitol. The reaction was stopped with 500 μl of 1 N HCl. The reaction mixtures were transferred to 20 ml scintillation vials and the [³H]-methylcatechol product was extracted with 10 ml of an organic scintillation cocktail. The samples were counted on a liquid scintillation counter.

Data were analyzed in Microsoft Excel to determine the femtomoles of [³H]-methylcatechol produced in the assay per 20 minutes (abbreviated fmol [³H]-methylcatechol/20 min) and were plotted in GraphPad Prism.

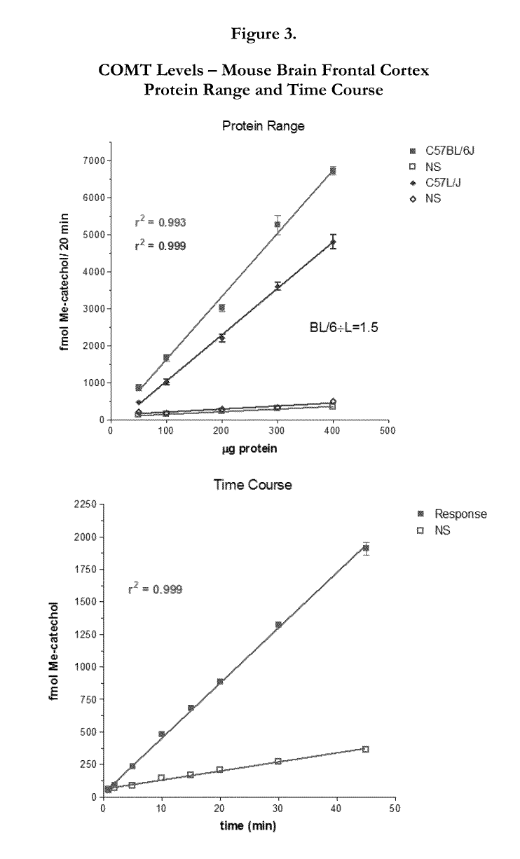

COMT activity was tested in frontal cortex homogenates from C57BL/6J and C57L/J mice over a wide range of protein concentrations (50-400 μg) and for a range of incubation times (1-45 min). With the inclusion of AdoHcy Nucleosidase (EC 3.2.2.9) in the assay, there was a linear relationship for both protein concentrations and for incubation time (FIG. 3). Due to feedback inhibition by S-adenosyl-L-homocysteine accumulation, these relationships were curvilinear for both variables without EC 3.2.2.9 (not shown). At standard incubation conditions, there was no feedback inhibition.

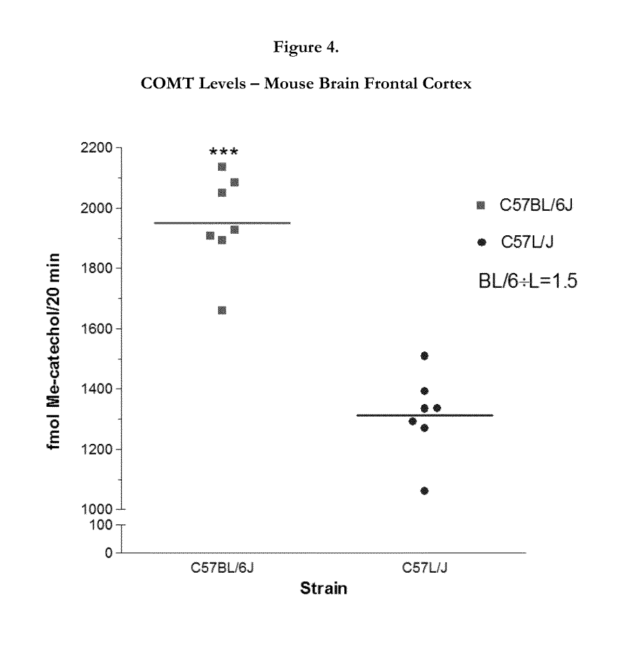

The initial comparison of COMT activity in frontal cortex samples from C57BL/6J and C57L/J strains indicated that there was 1.5 times greater activity in the C57BL/6J cortex (FIG. 4). This finding was demonstrated repeatedly. The determination of cortical COMT activity from seven cohorts of the C57BL/6J and C57L/J mice confirmed the result with highly significant differences between the two strains. C57BL/6J mice had COMT activity of 1951±60.20 N=7 (fmol/20 min) vs. 1314±51.42 N=7 for the C57L/J mice for a P value of P<0.001 in a two-tailed unpaired t test.

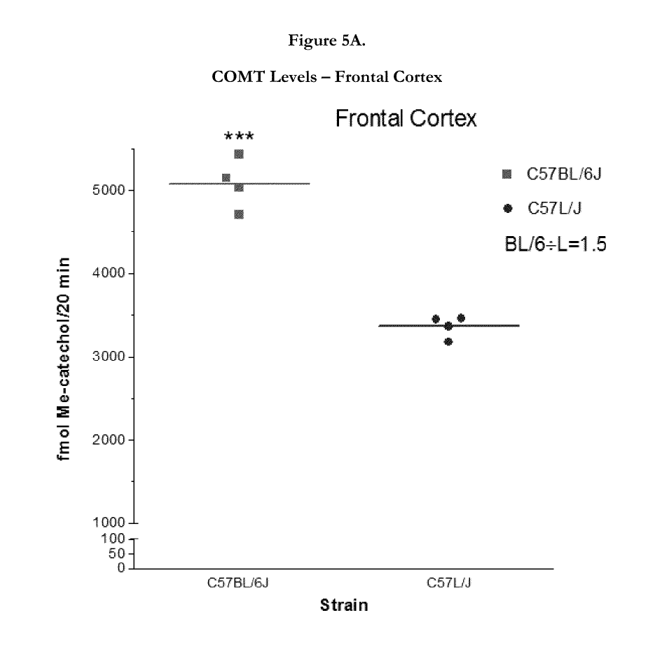

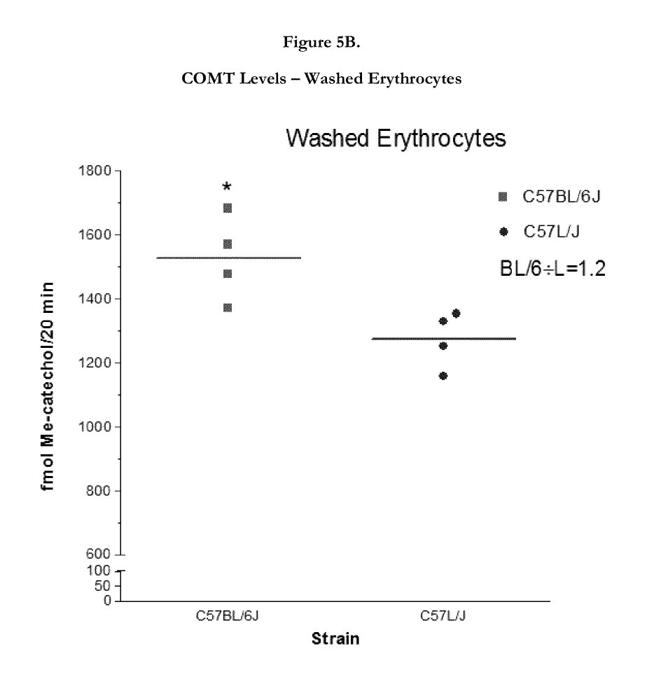

In a smaller study of four mice of each strain, there was again a highly significant difference of 1.5 times activity for the C57BL/6J frontal cortex vs. the C57L/J frontal cortex (FIG. 5A). For washed erythrocytes, the difference was less pronounced at 1.2 times activity for the C57BL/6J vs. the C57L/J, but the difference was still significant (P=0.019) (FIG. 5B).

The animal model described herein has significance in that it has the potential to serve as a surrogate for human single nucleotide polymorphisms at the Val¹⁵⁸Met locus of the COMT gene. Val/Val homozygotes have been shown to have 1.4-fold greater frontal cortex COMT activity than Met/Met homozygotes (Chen, et al., Am. J. Hum. Genet. 75:807-821, 2004). The 1.5-fold difference between C57BL/6J and C57L/J mice is highly significant and, as such, may be used to demonstrate the utility of treatment effect on COMT activity by alpha 7 nAChR agonists alone or in combination with antipsychotics and/or COMT inhibitors.

It is understood that the foregoing detailed description and accompanying examples are merely illustrative and are not to be taken as limitations upon the scope of the invention, which is defined solely by the appended claims and their equivalents.

What is claimed is:

1. A method of identifying a patient with schizophrenia as a candidate for effective treatment with a nicotinic acetylcholine receptor ligand modulator, the method comprising:
(a) obtaining a sample from the patient with schizophrenia;
(b) determining the identity of an allele of at least one single nucleotide polymorphism (SNP) locus associated with the catechol-O-methyltransferase (COMT) gene in the sample;
(c) determining the smoking status of the patient with schizophrenia;
(d) identifying the patient with schizophrenia as a candidate for effective treatment with the nicotinic alpha 7 receptor agonist based on the presence or absence of a particular SNP allele associated with the COMT gene in the sample and the smoking status of the patient with schizophrenia; and
(e) administering to the patient in need thereof an effective amount of a particular nicotinic acetylcholine receptor ligand modulator based upon result of step (d).

2. The method of claim 1, wherein the nicotinic acetylcholine receptor is α-7 nicotinic receptor.

3. A method of identifying a patient with schizophrenia with an increased likelihood of response to treatment with a nicotinic alpha 7 receptor agonist, the method comprising:
(a) obtaining a sample from the patient with schizophrenia;
(b) determining the identity of an allele of at least one single nucleotide polymorphism (SNP) locus associated with the catechol-O-methyltransferase (COMT) gene in the sample;
(c) determining the smoking status of the patient with schizophrenia; and
(d) identifying the patient with schizophrenia as having an increased likelihood of response to treatment with the nicotinic alpha 7 receptor agonist based on the presence or absence of a particular SNP allele associated with the COMT gene in the sample and the smoking status of the patient with schizophrenia.

4. The method of claims 1-3, wherein the presence of at least one SNP allele associated with the COMT gene in the patient with schizophrenia identifies the patient with schizophrenia as a candidate for effective treatment with the nicotinic alpha 7 receptor agonist.

5. A method of identifying and treating a patient with schizophrenia with an effective dosage of nicotinic alpha 7 receptor agonist, the method comprising:
(a) obtaining a sample from the patient with schizophrenia;
(b) determining the identity of an allele of at least one single nucleotide polymorphism (SNP) locus associated with the catechol-O-methyltransferase (COMT) gene in the sample;
(c) determining the smoking status of the patient with schizophrenia;
(d) identifying the patient with schizophrenia as a candidate for effective treatment with the nicotinic alpha 7 receptor agonist based on the presence or absence of a particular SNP allele associated with the COMT gene in the sample and the smoking status of the patient with schizophrenia; and
(e) administering an effective dosage of nicotinic alpha 7 receptor agonist to the patient with schizophrenia identified as being a candidate for effective treatment with the nicotinic alpha 7 receptor agonist.

6. The method of any one of the preceding claims, wherein the SNP associated with the COMT gene is located in the COMT gene or in a region surrounding the COMT gene.

7. The method of any one of the preceding claims, wherein the SNP associated with the COMT gene is located in the COMT gene.

8. The method of any one of the preceding claims, wherein the SNP is at least one of rs6269, rs4633, rs4680, and rs4818, or a SNP in linkage disequilibrium with at least one of the foregoing SNPs, or combinations thereof.

9. The method of any one of the preceding claims, wherein the SNP is at least one of rs6269, rs4633, rs4680, and rs4818, or combinations thereof.

10. The method of any one of claims 1-8, wherein the SNP is at least one of a SNP in linkage disequilibrium with at least one of rs6269, rs4633, rs4680, and rs4818, or combinations thereof.

11. The method of any one of the claims 1-9, wherein the presence of at least one of G/C or G/G for rs4818, A/A or G/A or rs4680, T/T or C/T for rs4633, G/G or A/G for rs6269 identifies the patient with schizophrenia as a candidate for effective treatment with nicotinic alpha 7 receptor agonist.

12. The method of any one of the preceding claims, wherein the patient with schizophrenia is a smoker.

13. The method of any one of the preceding claims, wherein the patient with schizophrenia is a non-smoker.

14. The method of any one of the preceding claims, wherein the nicotinic alpha 7 receptor agonist comprises a compound selected from the group consisting of (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide and salts thereof.

15. The method of any one of the preceding claims, wherein the nicotinic alpha 7 receptor agonist comprises (4s)-4-(5-phenyl-1,3,4-thiadiazol-2-yloxy)-1-azatricyclo[3.3.1.1^{3,7}]decane.

16. The method of any one of the preceding claims, wherein the effective dosage range of the nicotinic alpha 7 receptor agonist is about 10-25 mg/kg of body weight daily.

17. A method for monitoring the treatment of a patient suffering from schizophrenia, schizophreniform disorder or a related schizophrenia spectrum psychotic disorder, the method comprising:
(a) obtaining a sample from the patient wherein the patient is already under a treatment regimen of a particular nAChr ligand modulator;
(b) determining the identity of an allele of at least one single nucleotide polymorphism (SNP) locus associated with the COMT gene in the sample;
(c) determining the smoking status of the patient; and if necessary,
(d) modifying the course of treatment including administering a different nAChr ligand modulator based upon the presence or absence of particular SNPs associated with the COMT gene in the patient.

18. The method of claim 17, wherein the SNP associated with the COMT gene is located in the COMT gene or in a region surrounding the COMT gene.

19. The method of claim 17 or 18, wherein the SNP associated with the COMT gene is located in the COMT gene.

20. The method of any one of claims 17-19, wherein the SNP is at least one of rs6269, rs4633, rs4680, and rs4818, or a SNP in linkage disequilibrium with at least one of the foregoing SNPs, or combinations thereof.

21. The method of any one of claims 17-20, wherein the SNP is at least one of rs6269, rs4633, rs4680, and rs4818, or combinations thereof.

22. The method of any one of claims 17-20, wherein the SNP is at least one of a SNP in linkage disequilibrium with at least one of rs6269, rs4633, rs4680, and rs4818, or combinations thereof.

23. The method of any one of claims 17-21, wherein the presence of at least one of G/C or G/G for rs4818, A/A or G/A or rs4680, T/T or C/T for rs4633, G/G or A/G for rs6269 identifies the patient with schizophrenia as a candidate for effective treatment with nicotinic alpha 7 receptor agonist.

24. The method of any one of claims 17-23, wherein the patient with schizophrenia is a smoker.

25. The method of any one of claims 17-24, wherein the patient with schizophrenia is a non-smoker.

26. The method of any one of claims 17-25, wherein the nicotinic alpha 7 receptor agonist comprises a compound selected from the group consisting of (4s)-4-(5-phenyl-1,3,4-thiadiazol-2-yloxy)-1-azatricyclo[3.3.1.1^{3,7}]decane, N-[2-(pyridin-3-ylmethyl)-1-azabicyclo[2.2.2]oct-3-yl]-1-benzofuran-2-carboxamide, N-[(3R)-1-azabicyclo[2.2.2]oct-3-yl]-7-chloro-1-benzothiophene-2-carboxamide, (R)-7-chloro-N-(quinuclidin-3-yl)benzo[b]thiophene-2-carboxamide and salts thereof.

27. The method of any one of claims 17-26, wherein the nicotinic alpha 7 receptor agonist comprises (4s)-4-(5-phenyl-1,3,4-thiadiazol-2-yloxy)-1-azatricyclo[3.3.1.1^{3,7}]decane.

28. The method of any one of claims 17-27, wherein the effective dosage range of the nicotinic alpha 7 receptor agonist is about 10-25 mg/kg of body weight daily.
